(19) United States
(12) Patent Application Publication
(10) Pub. No.: US 2021/0275323 A1
(43) Pub. Date: Sep. 9, 2021

(54) DELIVERY DEVICES AND METHODS

(71) Applicant: Spinal Elements, Inc., Carlsbad, CA (US)

(72) Inventors: Matt Ono, San Diego, CA (US); Marc Yap, Carlsbad, CA (US); David Ortiz, Carlsbad, CA (US); Mort Albert, Carlsbad, CA (US); Jason Blain, Encinitas, CA (US)

(21) Appl. No.: 17/190,900

(22) Filed: Mar. 3, 2021

Related U.S. Application Data
(60) Provisional application No. 62/986,427, filed on Mar. 6, 2020.

(51) Int. Cl. A61F 2/46

(52) U.S. Cl. CPC A61F 2/4601; A61F 2002/4625; A61F 2002/4615; A61F 2/4611

(57) ABSTRACT

Various embodiments of delivery devices and methods for delivering material are provided. The delivery device can include a barrel support configured to couple with a barrel. The barrel can include a lumen configured to receive a cartridge. The delivery device can include a stock including a handle. The delivery device can include a break action configured to allow the barrel support and the stock to pivot. The method of delivering material to a surgical site can include inserting a tip of a barrel of a delivery device into a surgical site. The barrel can include a lumen. The barrel can be coupled to a barrel support. The barrel support can be coupled to a stock. The stock can include a handle. The method of delivering material to a surgical site can include actuating a break action to allow the stock to pivot relative to the barrel support.

### CROSS-REFERENCE TO RELATED APPLICATIONS

This application claims priority benefit to U.S. Provisional Patent Application No. 62/986427, filed Mar. 6, 2020, the entirety of which is hereby incorporated by reference herein.

### BACKGROUND

### Field

Some embodiments described herein relate generally to devices and methods for delivering material to a surgical site. Specifically, the disclosure relates to delivery devices, including those used for spinal fusion for delivering graft material, biologics, and/or other material to the disc space.

### Description of the Related Art

Advancing age, as well as injury, can lead to degenerative changes in the bones, discs, joints, and ligaments of the spine, producing pain and instability. Under certain circumstances, alleviation of the problems can be provided by performing spinal fusion. Spinal fusion is a surgical technique in which two or more vertebrae of the spinal column are fused together to eliminate the motion between the fused vertebrae. Spinal fusion is used to treat conditions where the spine exhibits instability. Spine instability may result from causes such as fracture, scoliosis, and spondylolisthesis, where one or more vertebrae move in a forward direction relative to the other vertebrae. Spinal fusion with discectomy is also performed for herniation of the discs. This surgery involves removal of the affected disc and fusion of the adjacent vertebrae. Traditionally, bone grafts have been used to fuse the vertebrae, but various types of vertebral implants have also been used.

The use of intervertebral implants and bone screw fixation systems for treating injuries to bones is well established. In most instances, an intervertebral implant is positioned between adjacent vertebrae in the disc space and secured to the bone. The intervertebral implant can be secured to the bone by bone screws or other similar fasteners inserted through holes in the intervertebral implant and into the bone itself. The screws are tightened so that the intervertebral implant holds the bone to be treated in place in order to insure proper healing.

In some spinal fusions, graft material can be inserted within the disc space to restore the height between adjacent vertebrae. In some procedures, other material such as biologics, medicines, and bone morphogenetic protein are inserted within the disc space. Notwithstanding the foregoing, there remains a need for improved methods and devices for delivering material to a surgical site.

### SUMMARY

In some embodiments, a delivery device is provided. The delivery device can include a barrel support configured to couple with a barrel. The delivery device can include a stock comprising a handle. In some embodiments, the stock is configured to pivot relative to the barrel support when the barrel is positioned at a surgical site. In some embodiments, the pivoting is configured to facilitate positioning a cartridge within a lumen of the barrel.

In some embodiments, the barrel support comprises a rotation knob configured to allow the barrel to rotate relative to the barrel support. In some embodiments, the delivery device can include a horizontal pivot pin configured to allow upward and downward motion of the stock relative to the barrel support. In some embodiments, the delivery device can include a vertical pivot pin configured to allow side to side motion of the stock relative to the barrel support. In some embodiments, the delivery device can include a trigger configured to allow the stock to pivot. In some embodiments, the delivery device can include a slide mechanism configured to allow the stock to pivot. In some embodiments, the delivery device can include at least one pin configured to slide within a groove of a guide when the stock pivots. In some embodiments, the delivery device can include an ejector flange configured to slide relative to a flange of a cartridge. In some embodiments, the delivery device can include a pin configured to slide out of an interference fit with a lumen of the barrel support to allow the stock to pivot. In some embodiments, the delivery device can include a stop configured to slide out of engagement with the barrel support to allow the stock to pivot. In some embodiments, the delivery device can include a pawl and a ratchet rod to advance material through the cartridge. In some embodiments, the delivery device can include a slap hammer configured to couple with the stock. In some embodiments, the delivery device can include a trigger to advance material through the cartridge.

In some embodiments, a method of delivering material to a surgical site is provided. The method can include inserting a tip of a barrel of a delivery device into a surgical site. In some embodiments, the barrel is coupled to a barrel support. In some embodiments, the barrel support is coupled to a stock comprising a handle. The method can include pivoting the stock relative to the barrel support when the barrel is positioned at the surgical site. The method can include inserting a cartridge within a lumen of the barrel when the stock is pivoted.

In some embodiments, the method can include removing the cartridge from the lumen of the barrel. In some embodiments, the method can include inserting a second cartridge into the lumen of the barrel. In some embodiments, pivoting the stock relative to the barrel support further comprises pulling a trigger with the index finger of a hand of a user. In some embodiments, the method can include holding the handle with the hand of the user while actuating the trigger. In some embodiments, the stock is pivoted upward and downward relative to the barrel support. In some embodiments, the stock is pivoted side to side relative to the barrel support.

### BRIEF DESCRIPTION OF THE DRAWINGS

The structure and method of using delivery devices will be better understood with the following detailed description of embodiments, along with the accompanying illustrations, in which:

FIG. 1 is a perspective view of an embodiment of a delivery device.

FIG. 2 is a top view of the delivery device of FIG. 1.

FIG. 3 is a side view of the delivery device of FIG. 1.

FIG. 4 is a cross-sectional side view of the delivery device of FIG. 1.

FIG. 5 is an exploded perspective view of the delivery device of FIG. 1.

FIG. 6 is a perspective view of the delivery device of FIG. 1 upon activation of a break action.

FIG. 7 is a cross-sectional side view of the delivery device of FIG. 1 upon activation of the break action.

FIG. 8 is a perspective view of an embodiment of a delivery device.

FIG. 9 is a top view of the delivery device of FIG. 8.

FIG. 10 is a side view of the delivery device of FIG. 8.

FIG. 11 is a cross-sectional side view of the delivery device of FIG. 8.

FIG. 12 is an exploded perspective view of the delivery device of FIG. 8.

FIG. 13 is a perspective view of the delivery device of FIG. 8 upon activation of a break action.

FIG. 14 is a cross-sectional side view of the delivery device of FIG. 8 upon activation of the break action.

FIG. 15 is a perspective view of an embodiment of a delivery device.

FIG. 16 is a top view of the delivery device of FIG. 15.

FIG. 17 is a side view of the delivery device of FIG. 15.

FIG. 18 is a cross-sectional side view of the delivery device of FIG. 15.

FIG. 19 is an exploded perspective view of the delivery device of FIG. 15.

FIG. 20 is a perspective view of the delivery device of FIG. 15 upon activation of a break action.

FIG. 21 is a cross-sectional side view of the delivery device of FIG. 15 upon activation of the break action.

FIG. 22 is a perspective view of an embodiment of a delivery device.

FIG. 23 is a top view of the delivery device of FIG. 22.

FIG. 24 is a side view of the delivery device of FIG. 22.

FIG. 25 is a cross-sectional side view of the delivery device of FIG. 22.

FIG. 26 is an exploded perspective view of the delivery device of FIG. 22.

FIG. 27 is a perspective view of the delivery device of FIG. 22 upon activation of a break action.

FIG. 28 is a cross-sectional side view of the delivery device of FIG. 22 upon activation of the break action.

FIG. 29 is a perspective view of an embodiment of a delivery device.

FIG. 30 is a top view of the delivery device of FIG. 29.

FIG. 31 is a side view of the delivery device of FIG. 29.

FIG. 32 is a cross-sectional side view of the delivery device of FIG. 29.

FIG. 33 is an exploded perspective view of the delivery device of FIG. 29.

FIG. 34 is a perspective view of the delivery device of FIG. 29 upon activation of a break action.

FIG. 35 is a cross-sectional side view of the delivery device of FIG. 29 upon activation of the break action.

FIG. 36 is a perspective view of an embodiment of a delivery device.

FIG. 37 is a top view of the delivery device of FIG. 36.

FIG. 38 is a side view of the delivery device of FIG. 36.

FIG. 39 is a cross-sectional side view of the delivery device of FIG. 36.

FIG. 40 is an exploded perspective view of the delivery device of FIG. 36.

FIG. 41 is a perspective view of the delivery device of FIG. 36 upon activation of a break action.

FIG. 42 is a cross-sectional side view of the delivery device of FIG. 36 upon activation of the break action.

### DETAILED DESCRIPTION

Although certain preferred embodiments and examples are disclosed below, it will be understood by those in the art that the invention extends beyond the specifically disclosed embodiments and/or uses of the invention and obvious modifications and equivalents thereof. Thus, it is intended that the scope of the invention herein disclosed should not be limited by the particular disclosed embodiments described below.

Delivery devices can be utilized to deliver any substance or material to a surgical site. As used in this specification, a substance or material can include any biologic and/or chemical substance or material, including, but not limited to, biologics, medicine, bone morphogenetic protein, and/or a bone graft, including, but not limited to, autograft, allograft, xenograft, alloplastic graft, a synthetic graft, and/or combinations of one or more biologics, medicine, bone morphogenetic protein, and/or a bone graft. As used herein, substance and material can be used interchangeably throughout. While exemplary references are made with respect to vertebra, in some embodiments any surgical site can be accessed for the delivery of substances. While specific reference may be made to a vertebra and/or subset and/or grouping of vertebrae, it is understood that any surgical site within the body of the patient can be used.

As disclosed herein, the delivery devices can be opened to allow insertion or removal of a cartridge containing the substance. The delivery devices can be opened via a pivoting connection. The delivery device can include a hinged opening at a location near where a stock meets a barrel. By opening the delivery device, the cartridge can be more easily loaded and removed. A separate action from the break action may be required for advancing the material through the cartridge. In some embodiments, the delivery devices can be opened during a surgical procedure without removing the tip of the delivery device from the surgical site. By opening the delivery device during the procedure, a spent cartridge can be removed and a new cartridge can be loaded. A spent cartridge can include any amount of substance or no remaining substance.

Although referred to as delivery devices, these devices need not be used for the delivery of substances, but the break action can be used for any surgical tool to allow loading and unloading of an object, or to generally provide an opening into a pathway for any of a number of purposes.

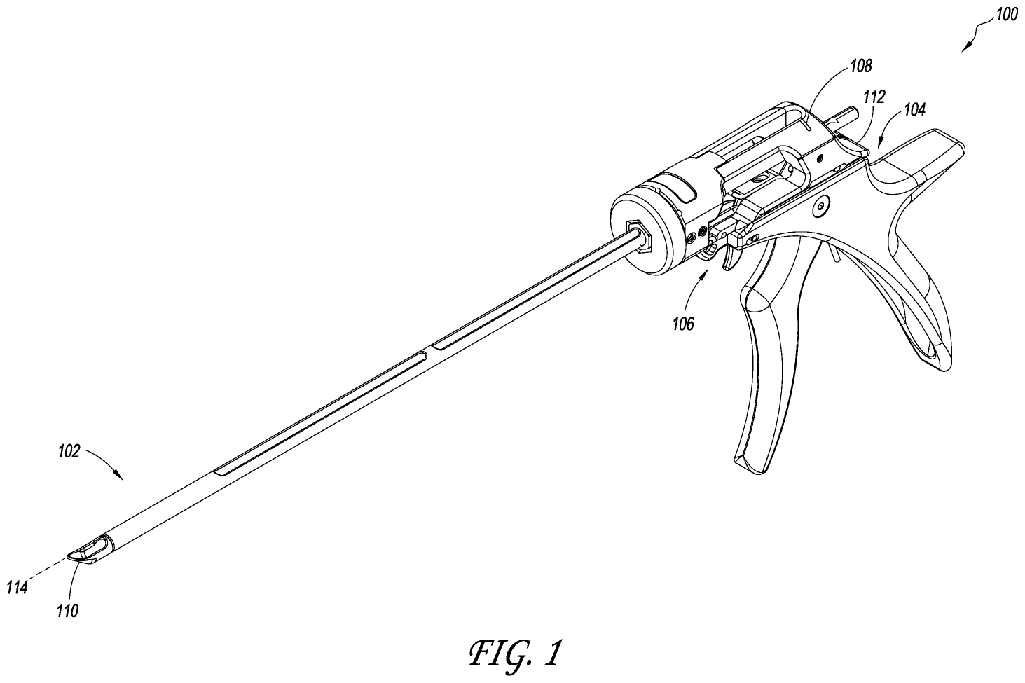

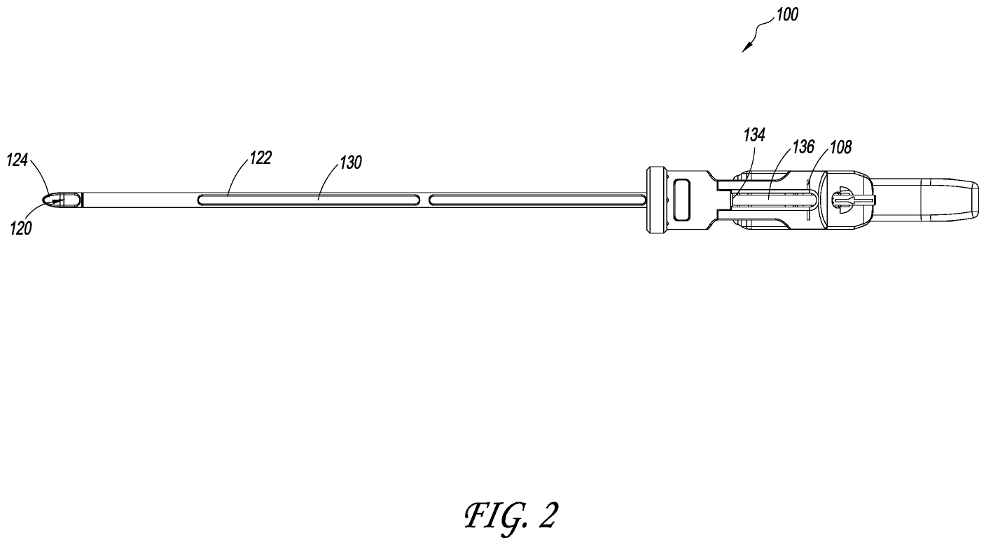

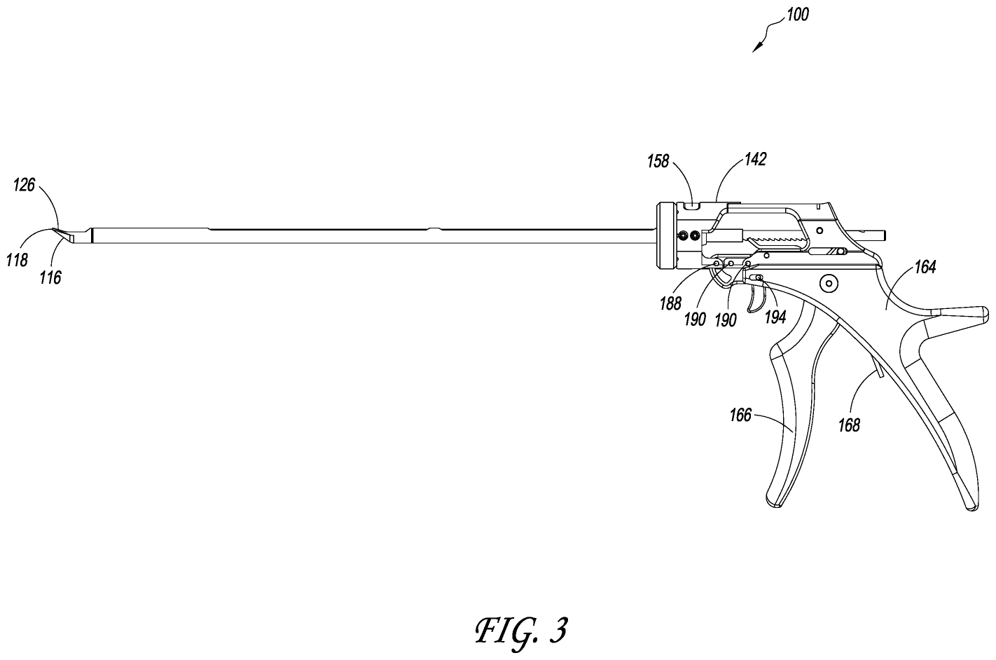

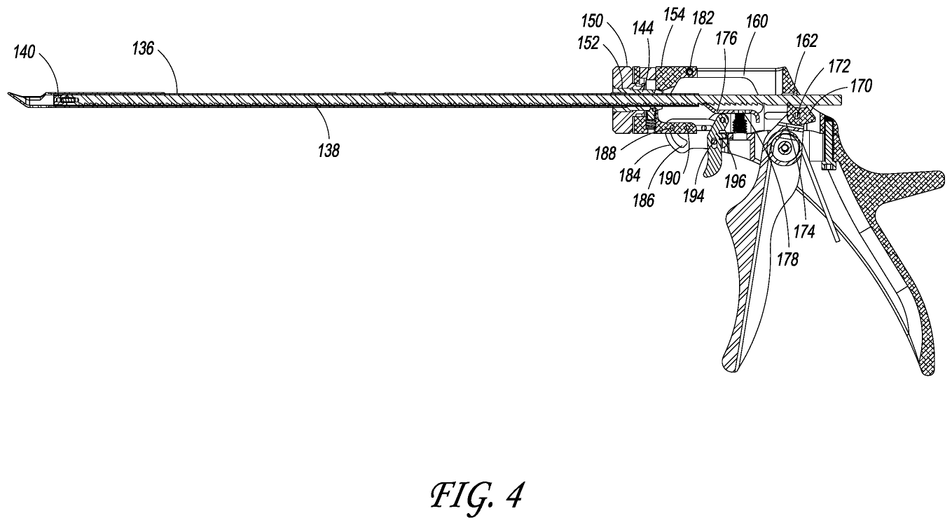

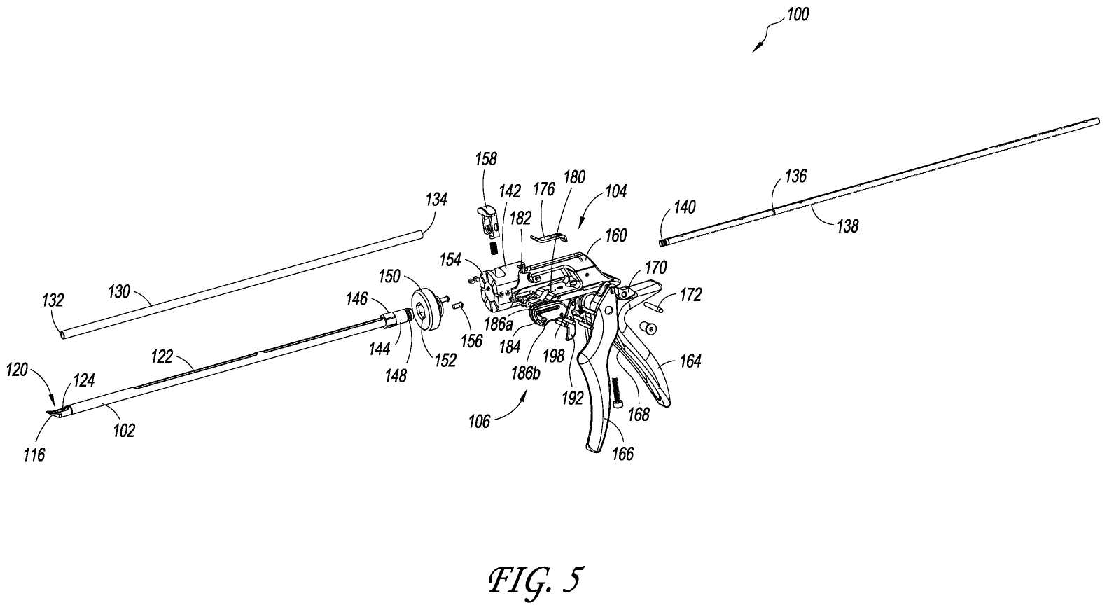

FIG. 1 shows a perspective view of an embodiment of a delivery device 100. FIG. 2 shows a top view of an embodiment of the delivery device 100. FIG. 3 shows a side view of the delivery device 100. FIG. 4 shows a cross-sectional view of the delivery device 100. FIG. 5 is an exploded perspective view of the delivery device 100.

As shown in FIGS. 1-5, the delivery device 100 can comprise a barrel 102. The barrel 102 can form an elongate pathway. The delivery device 100 can include a stock 104. The stock 104 can allow a user to manipulate the delivery device 100, for instance by advancing a material within the barrel 102 for delivery. The delivery device 100 can include a break action 106. The break action 106 can be a connection between the barrel 102 and the stock 104. The break action 106 can include a hinge joint that joins two parts of the delivery device 100. As described herein, the delivery device 100 can break in plane. The delivery device 100 can break in a forward-backward action.

The delivery device 100 can include a distal end 110 and a proximal end 112. The delivery device 100 can have a longitudinal axis 114 extending between the distal end 110 and the proximal end 112. The longitudinal axis 114 can extend along the barrel 102. The barrel 102 can be disposed near the distal end 110 of the device. The barrel 102 can have an elongate shape. The barrel 102 can be straight or linear. In some embodiments, the barrel 102 can have a non-linear shape such as a curved pathway.

The distal end of the barrel 102 can form the distal end 110 of the delivery device 100. The barrel 102 can include an insertion surface 116. In some embodiments, the insertion surface 116 can be angled, for instance, 15 degrees from the longitudinal axis 114, 30 degrees from the longitudinal axis 114, 45 degrees from the longitudinal axis 114, 60 degrees from the longitudinal axis 114, 90 degrees from the longitudinal axis 114, or any range of the foregoing values. In some embodiments, the insertion surface 116 can be flat or blunt. The insertion surface 116 can include one or more rounded edges. The insertion surface 116 can include an atraumatic tip 118. The atraumatic tip 118 can include a rounded point to facilitate insertion of the distal end 110 to the surgical site.

The barrel 102 can include a lumen 120. The lumen 120 can extend along the longitudinal axis 114. The barrel 102 can form a tubular structure. The barrel 102 can include one or more openings 122 extending from an outer surface of the barrel 102 to the lumen 120. The illustrated embodiment includes two openings 122, but any number of openings 122 are contemplated. The barrel 102 can include one opening 122, two openings 122, three openings 122, four openings 122, five openings 122, or any range of the foregoing values. Two or more openings 122 can be axially aligned. Two or more openings 122 can be circumferentially offset. The openings 122 can include any shape. The openings 122 can be oval or rounded. The openings 122 can be elongate. The openings 122 can be spaced away from the distal end 110. The openings 122 can allow the contents of the lumen 120 to be viewed by the user.

The barrel 102 can include a delivery opening 124. The delivery opening 124 can be the same shape as the opening 122. The delivery opening 124 can be a different shape as the opening 122. The barrel 102 can include the delivery opening 124 extending from an outer surface of the barrel 102 to the lumen 120. The illustrated embodiment includes one delivery opening 124, but any number of delivery openings 124 are contemplated. The barrel 102 can include one delivery opening 124, two delivery openings 124, three delivery openings 124, four delivery openings 124, five delivery openings 124, or any range of the foregoing values. The delivery opening 124 and the opening 122 can be axially aligned. The delivery opening 124 and the opening 122 can be circumferentially offset. The delivery opening 124 can include any shape. The delivery opening 124 can be oval or rounded. The delivery opening 124 can be elongate. The delivery opening 124 can be disposed closer to the distal end 110 than the proximal end 112. The delivery opening 124 can allow material to be expelled from the lumen 120. The barrel 102 can include a lip 126. The lip 126 can direct the flow of material. The lip 126 can extend beyond the circumference of the barrel 102.

In some embodiments, the barrel 102 can include one or more discrete sections. The barrel 102 can include one or more removable sections. In some embodiments, the barrel 102 can include a first section including the delivery opening 124. In some embodiments, the barrel 102 can include a second section including the one or more openings 122. In some embodiments, the sections include the same material. In some embodiments, the sections include different materials, such as the first section comprising a more rigid material than the second section. In some embodiments, the barrel 102 is monolithically or integrally formed. In some embodiments, sections of the barrel 102 are separately formed. The sections of the barrel 102 can be joined by means such as adhesive, welding, threaded connection, pins, detents, interference fit, snap fit, or any other attachment means.

The lumen 120 of the barrel 102 is sized to receive a cartridge 130. The cartridge 130 can contain material to be delivered to the surgical site. The cartridge 130 can be loaded with material by the user. The cartridge 130 can be loaded with material during manufacturing of the cartridge 130. In some embodiments, the cartridge 130 is disposable. In some embodiments, the cartridge 130 is reusable. In some embodiments, a kit is provided to the user. One or more cartridges 130 can be included in the kit. In some embodiments, the delivery device 100 is provided in a kit without the cartridge 130. The cartridge 130 can form a tubular structure. The cartridge 130 can include a distal open end 132 and a proximal open end 134. The distal open end 132 can be positioned near the delivery opening 124 when the cartridge 130 is disposed within the lumen 120 of the barrel 102. During use, material is moved toward the distal open end 132 of the cartridge 130.

The cartridge 130 is sized to receive a ratchet rod 136. The ratchet rod 136 can form an elongate structure. The ratchet rod 136 can include a series of gears 138. The gears 138 can extend along the entire ratchet rod 136 or a portion thereof. The gears can extend along 50% of the length of the ratchet rod 136, 60% of the length of the ratchet rod 136, 70% of the length of the ratchet rod 136, 80% of the length of the ratchet rod 136, 90% of the length of the ratchet rod 136, 100% of the length of the ratchet rod 136, or any range of the foregoing values. The ratchet rod 136 can include a plunger 140. The plunger 140 can push the contents of the cartridge 130 distally. The plunger 140 can push the contents of the cartridge 130 toward the delivery opening 124. The plunger 140 can be sized to scrape the inside of the cartridge 130 when the ratchet rod 136 is advanced. In some embodiments, the plunger 140 and the ratchet rod 136 are integrally formed. In some embodiments, the plunger 140 and the ratchet rod 136 are separate components. The plunger 140 and the ratchet rod 136 can be joined by means such as adhesive, welding, threaded connection, pins, detents, interference fit, snap fit, or any other attachment means.

The delivery device 100 can include a barrel support 142. The barrel support 142 can couple to the proximal end of the barrel 102. In some embodiments, the barrel 102 and the barrel support 142 can include one or more discrete components. The barrel 102 and the barrel support 142 can be separately formed. The barrel 102 can be removable from the barrel support 142. In some embodiments, the barrel 102 and the barrel support 142 are monolithically or integrally formed.

The cartridge 130 can extend into the barrel support 142 from the barrel 102 when the cartridge 130 is disposed within the barrel 102. The cartridge 130 can extend proximally beyond the proximal end of the barrel 102. In the illustrated embodiment, the ratchet rod 136 can extend proximally beyond the proximal end of the barrel 102. The ratchet rod 136 can extend proximally beyond the proximal open end 134 of the cartridge 130. The ratchet rod 136 can extend through the barrel support 142.

The proximal end of the barrel 102 can be coupled to a hub 144. In some embodiments, the barrel 102 and the hub 144 can include one or more discrete components. The barrel 102 and the hub 144 can be separately formed. The barrel 102 can be removable from the hub 144. The proximal end of the barrel 102 and the hub 144 can be joined by means such as adhesive, welding, threaded connection, pins, detents, interference fit, snap fit, or any other attachment means. In some embodiments, the barrel 102 and the hub 144 are monolithically or integrally formed. The hub 144 can include a keyed section 146. The keyed section 146 can ensure that the barrel 102 is oriented relative to other components of the delivery device 100. The hub 144 can include a groove 148. The groove 148 can ensure retention of the hub 144 by the barrel support 142.

The barrel support 142 can include a rotation knob 150. The rotation knob 150 can allow the user to rotate the barrel 102. Rotation of the barrel 102 can position the delivery opening 124 in two or more circumferential positions. The rotation knob 150 includes a keyed opening 152. The keyed opening 152 can be sized to accept the keyed section 146 of the hub 144. Rotation of the rotation knob 150 can cause a corresponding rotation of the hub 144, and thus a corresponding rotation of the barrel 102. The barrel support 142 can include a base 154 configured to interact with the rotation knob 150. The user can rotate the barrel 102, the hub 144, and the rotation knob 150 relative to the base 154. The barrel support 142 can include one or more ball plungers 156, shown in exploded view in FIG. 5. The ball plungers 156 can extend from the rotation knob 150. The ball plungers 156 can interact with the base 154 to lock the rotational position of the rotation knob 150 relative to the base 154. The barrel support 142 can include a button 158. The button 158 can allow the hub 144 to disengage from the rotation knob 150 to remove barrel 102. The user can depress the button 158. As the button 158 moves downward, the button 158 disengages the groove 148 of the hub 144 to allow the hub 144 to disengage from the rotation knob 150.

The rotation knob 150 can allow discrete rotational positions. In the illustrated embodiment, the rotation knob 150 allows six discrete circumferential positions of the barrel 102. Other numbers of discrete positions are contemplated including two discrete positions, three discrete positions, four discrete positions, five discrete positions, six discrete positions, seven discrete positions, eight discrete positions, nine discrete positions, ten discrete positions, or any range of the foregoing values. Other forms of rotation knobs are contemplated. In some embodiments, the rotation knob allows infinite rotational positions. In some embodiments, the barrel 102 cannot rotate relative to the barrel support 142.

The delivery device 100 can include the stock 104. The stock 104 can allow the user to manipulate the delivery device 100, for instance by advancing material through the cartridge 130 and through the delivery opening 124 of the barrel 102. The stock 104 can include a base 160. The base 160 of the stock 104 can interact with the base 154 of the barrel support 142 to allow pivoting action. The base 160 of the stock 104 can include a lumen 162. The lumen 162 is sized to accept the ratchet rod 136. In some embodiments, the ratchet rod 136 can extend from the proximal end of the stock 104.

The stock 104 can include a handle 164. The handle 164 can be ergonomically shaped to fit the hand of the user. The handle 164 can be shaped for right hand use. The handle 164 can be shaped for left hand use. The handle 164 can be shaped for ambidextrous use. In some embodiments, the base 160 of the stock 104 and the handle 164 are monolithically or integrally formed. In some embodiments, the base 160 of the stock 104 and the handle 164 are separately formed. The base 160 of the stock 104 and the handle 164 can be joined by means such as adhesive, welding, threaded connection, pins, detents, interference fit, snap fit, or any other attachment means.

The delivery device 100 can include a trigger 166. The trigger 166 can be utilized to deliver material. The trigger 166 can be ergonomically shaped to fit the hand of the user. The trigger 166 can be shaped for right hand use. The trigger 166 can be shaped for left hand use. The trigger 166 can be shaped for ambidextrous use. The delivery device 100 can include a spring 168. The spring 168 can bias the trigger 166. The user can compress the spring 168 when the trigger 166 is squeezed toward the handle 164. The spring 168 can bias the trigger 166 away from the handle 164 when the user releases the trigger 166.

The delivery device 100 can include a pawl 170. The delivery device 100 can include a pivot pin 172. The pivot pin 172 can extend through the trigger 166. The pawl 170 can be disposed within a groove 174 of the trigger 166. As the trigger 166 is squeezed toward the handle 164, the groove 174 of the trigger 166 interacts with the pawl 170 to cause the pawl 170 to rotate relative to the pivot pin 172. The pawl 170 engages a gear 138 of the ratchet rod 136. Each compression of the trigger 166 causes the pawl 170 to engage the next successive gear 138. During the compression of the trigger 166, the pawl 170 slides proximally along the gear 138, then the pawl 170 disengages the gear 138, and engages the next proximal gear 138. Another squeeze of the trigger 166 causes the pawl 170 to slide proximally along proximal gear 138, disengage proximal gear 138, and engages the next proximal gear 138. The trigger 166 causes the ratchet rod 136 to advance distally. The pawl 170 is guided by a horizontal slot and a vertical internal slot of the trigger 166. The pawl 170 cams out on the trigger 166 when advanced forward and advances the ratchet rod 136. When the pawl 170 moves backwards, the pawl 170 does not cam on the trigger 166 and rocks along the gears 138 of the ratchet rod 136 as the pawl 170 walks backwards without engaging the ratchet rod 136. The pawl 170 is dimensioned to a height that must rock between gears 138 when moving backwards and always engages a gear 138 when moving forwards.

The delivery device 100 can include a rocker 176. The delivery device 100 can include a pivot pin 178. The pivot pin 178 can extend through the base 160 of the stock 104. The rocker 176 can be disposed within a groove 180 of the base 160 of the stock 104. The rocker 176 can limit or prevent proximal motion of the ratchet rod 136. The rocker 176 can allow movement of the ratchet rod 136 in a distal direction. As the pawl 170 advances the ratchet rod 136, the rocker 176 rocks downward and out of engagement with the ratchet rod 136. The rocker 176 teeters along the ratchet rod 136. When the pawl 170 engages the proximal gear 138, the rocker 176 engages a gear 138. Upon additional distal motion of the ratchet rod 136, the rocker 176 engages another gear 138. The rocker 176 acts as a stop against the gear 138, thereby limiting or preventing proximal motion of the ratchet rod 136.

The delivery device 100 can include a break action 106. The break action 106 can be considered a connection between the barrel 102 and the stock 104. The break action 106 can be considered a connection between the base 154 of the barrel support 142 and the base 160 of the stock 104. The break action 106 allows a portion of the delivery device 100 to pivot relative to another portion of the delivery device 100. In some methods of use, the break action 106 allows for loading and unloading of the cartridge 130 within the barrel 102.

The break action 106 can include a pivot pin 182. In some embodiments, the pivot pin 182 allows the barrel support 142 and the stock 104 to separate. In some embodiments, the pivot pin 182 can include ball plungers to allow the barrel support 142 and the stock 104 to separate. In some embodiments, the pivot pin 182 can be a pivot point to allow the stock 104 to pivot relative to the barrel support 142 if there is a release element to allow separation of the stock 104 from the barrel support 142 on the underside of the delivery device 100. The pivot pin 182 can be any pivot that allows the delivery device 100 to pivot. In some embodiments, the pivot pin 182 allows the barrel support 142 and the stock 104 to pivot but not separate. Alternative embodiments to allow variations in how the delivery device 100 opens are contemplated. The pivot pin 182 can be disposed between the base 154 of the barrel support 142 and the base 160 of the stock 104. The pivot pin 182 can be disposed horizontal to the longitudinal axis 114. In some embodiments, the pivot pin 182 can be disposed perpendicular to the longitudinal axis 114. In some embodiments, the pivot pin 182 can be disposed parallel to the longitudinal axis 114. The pivot pin 182 can be disposed to allow the base 160 of the stock 104 to pivot upward or downward relative to the base 154 of the barrel support 142. In some embodiments, the pivot pin 182 can be disposed to allow the base 160 of the stock 104 to pivot left or right relative to the base 154 of the barrel support 142. In some embodiments, the pivot pin 182 can be disposed to allow the base 160 of the stock 104 to revolve outward or inward relative to the base 154 of the barrel support 142.

The break action 106 can include a guide 184. The guide 184 can include a groove 186. The groove 186 can include a straight portion 186a. The groove 186 can include a curved portion 186b. The delivery device 100 can include a pivot pin 188. The pivot pin 188 can extend through the base 154 of the barrel support 142. The pivot pin 188 can be disposed within the groove 186 of the guide 184. When the pivot pin 188 is disposed within the straight portion of the groove 186a, the delivery device 100 cannot pivot. When the pivot pin 188 is disposed within the curved portion of the groove 186b, the delivery device 100 can pivot. The curved portion of the groove 186b can be a cammed slot. The guide 184 can be a release mechanism. The guide 184 can be spring loaded in a distal direction. The guide 184 can be a spring-loaded release mechanism.

The guide 184 can be coupled to the base 160 of the stock 104. The base 160 of the stock 104 and the guide 186 can form a sliding connection. One or more pins 190 can extend through the base 160 of the stock 104 and the straight portion of the groove 186a. One or more pins 190 can slide along the straight portion of the groove 186a as the guide 184 moves distally and proximally.

The break action 106 can include a break trigger 192. The delivery device 100 can include a pivot pin 194. The break trigger 192 can include a groove 196. The pivot pin 194 can be disposed within the groove 196 of the break trigger 192. The guide 184 can include a secondary groove 198. The pivot pin 194 can be disposed within the secondary groove 198 of the guide 184. The break action is released by pulling the break trigger 192 with the user's index finger. The break trigger 192 moves guide 184 backwards until the front pin 188 reaches the curved portion of the groove 186b, and allows the front half of the delivery device 100 to pivot in the curved portion of the groove 186b.

The break trigger 192 is pulled proximally. As the break trigger 192 is squeezed, the pivot pin 194 moves proximally. The pivot pin 194 can be disposed within the secondary groove 198 of the guide 184. The break trigger 192 overcomes the biasing force of the spring-loaded guide 184. The guide 184 moves proximally as the break trigger 192 is squeezed. The pivot pin 188 and the one or more pins 190 are disposed within the groove 186 of the guide 184. The one or more pins 190 move along the straight portion of the groove 186a. The pivot pin 188 moves from the straight portion of the groove 186a to be aligned with the curved portion of the groove 186b. In some methods, the user can rotate the barrel support 142 relative to the stock 104 as the pivot pin 188 moves along the curved portion of the groove 186b. The user can rotate the barrel support 142 about the pivot pin 182. In some methods, the user can rotate the stock 104 relative to the barrel support 142 as the pivot pin 188 moves along the curved portion of the groove 188b. The user can rotate the stock 104 about the pivot pin 182.

The delivery device 100 can include additional features. The base 160 of the stock 104 can include one or more markings 108. The markings 108 can indicate the location for a measurement for the ratchet rod 136. The ratchet rod 136 can include a scale (not shown). Aligning the markings with the scale of the ratchet rod 136 can provide information to the user about the advancement of the ratchet rod 136. The markings 108 can provide a measurement of volume of dispensed material.

The delivery device 100 is designed to place material at a surgical site. In some methods of use, the material is graft material. The graft material can be bone graft, including, but not limited to, autograft, allograft, xenograft, alloplastic graft, and/or a synthetic graft. In some methods, the bone graft can be inserted into the surgical site to restore the height between adjacent structures such as vertebral bodies. In some methods of use, the material is biologics. In some methods of use, the material is bone morphogenetic protein.

The delivery device 100 is designed to be inserted into a surgical site. In some methods of use, the surgical site is the disc space between adjacent vertebrae. In some methods of use, the surgical site is between articular processes. In some methods of use, the surgical site is between spinous processes. In some methods of use, the surgical site is between transverse processes. In some methods of use, the surgical site is between adjacent bones. In some methods of use, the surgical site is between bone fragments. In some methods of use, the surgical site is within a joint. In some methods of use, the surgical site is within a joint capsule. In some methods of use, the surgical site is within a disc space. In some methods of use, the surgical site is any surgical site within the body of a patient.

FIGS. 1-5 are views of the delivery device 100 in a first position. The delivery device 100 can be inserted into the surgical site in the first position. In the first position, the base 154 of the barrel support 142 is generally aligned with the base 160 of the stock 104. In some embodiments, the barrel 102 can be coupled with the barrel support 142. The barrel 102 can be coupled to the hub 144. The barrel 102 can be rotated relative to the barrel support 142. The barrel 102 is generally coaxial with the base 160 of the stock 104. The guide 184 is in a distal position. The pivot pin 188 is disposed within the straight portion of the groove 186a. When the guide 184 is in the distal position, the delivery device 100 cannot rotate. In some methods of use, the delivery device 100 can be impacted with a tool. The delivery device can be driven with a hammer. A force can be applied to drive the delivery device 100 into the surgical site.

In some methods of use, the cartridge 130 is loaded into the barrel 102 prior to the delivery device 100 being inserted into the surgical site. In some methods of use, the ratchet rod 136 is loaded into the barrel 102 prior to the delivery device 100 being inserted into the surgical site. In some methods of use, the cartridge 130 is loaded before the delivery device 100 reaches the surgical site. In some methods of use, the ratchet rod 136 is loaded before the delivery device 100 reaches the surgical site. In some methods of use, the break action is used before the delivery device 100 is positioned. In some methods of use, the break action 106 can be utilized before entry into the surgical site. In some methods of use, the cartridge 130 is not loaded into the barrel 102 when the delivery device 100 is being inserted into the surgical site. In some methods of use, the ratchet rod 136 is not loaded into the barrel 102 when the delivery device 100 is being inserted into the surgical site. In some methods of use, the cartridge 130 is loaded after the delivery device 100 reaches the surgical site. In some methods of use, the ratchet rod 136 is loaded after the delivery device 100 reaches the surgical site. In some methods of use, the break action is used after the delivery device 100 is positioned. In some methods of use, the break action 106 can be utilized after entry into the surgical site.

The delivery device 100 can include the break trigger 192. As the break trigger 192 is squeezed, the pivot pin 194 is guided by the break trigger 192 and moves proximally. The pivot pin 194 is also disposed within the secondary groove 198 of the guide 184. As the break trigger 192 is squeezed, the guide 184 moves proximally. As the break trigger 192 is squeezed, the pivot pin 188 slides within the straight portion of the groove 186a of the guide 184. As the break trigger 192 is squeezed, the pivot pin 188 moves from the straight portion of the groove 186a to be aligned with the curved portion of the groove 186b.

Once the pivot pin 188 is aligned with the curved portion of the groove 186b, the user can rotate the barrel support 142 and the stock 104 relative to each other. The pivot pin 188 moves along the curved portion of the groove 186b and the delivery device is pivoted. The user can pivot the stock 104 about the barrel support 142. The delivery device 100 opens in line with the barrel 102. The delivery device 100 has a break action function. The break action 106 allows the barrel support 142 and the stock 104 to pivot relative to each other.

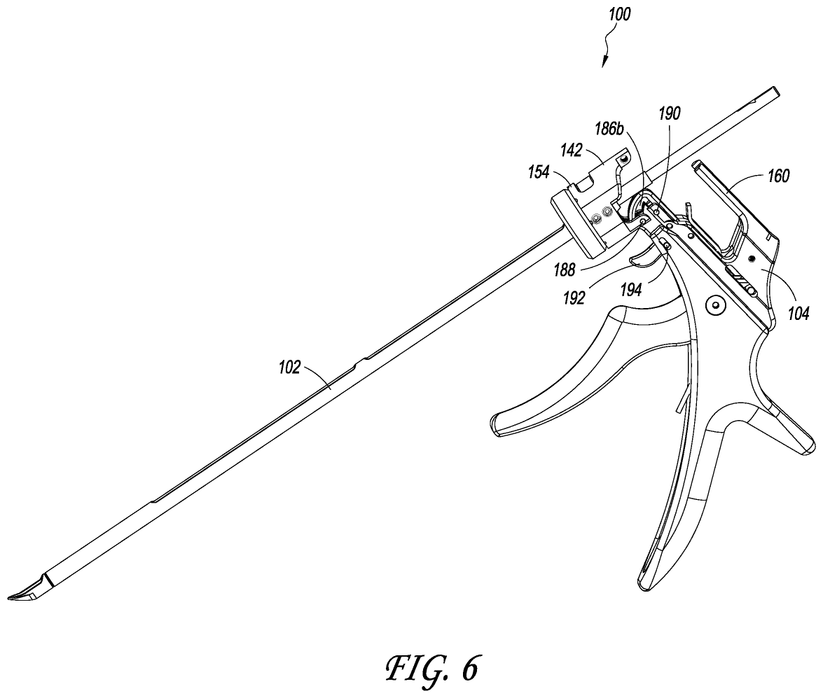

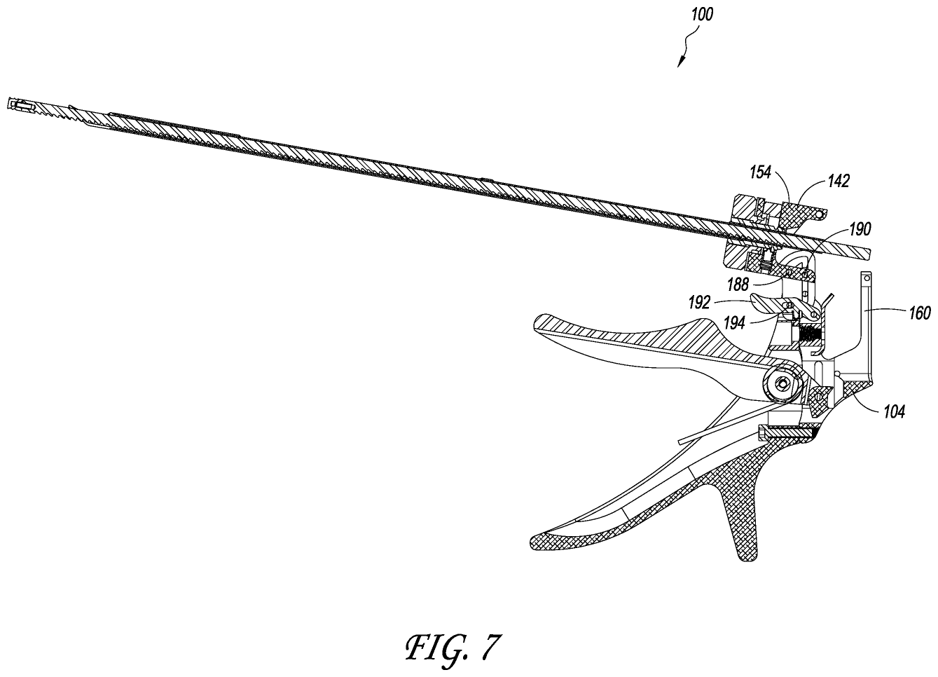

FIGS. 6-7 are views of the delivery device 100 in a second position. FIG. 6 is a perspective view of the delivery device 100 with the break action 106 activated. FIG. 7 is a cross-sectional view of the delivery device 100 with the break action 106 activated. Once the barrel support 142 and the stock 104 are pivoted, the cartridge 130 can be easily loaded into or removed from the barrel 102. The cartridge 130 can be dropped downward into the barrel 102 or removed upward from the barrel 102. The user can visualize the opening of the barrel 102 when the delivery device 100 is pivoted. The user can insert the cartridge 130 into the barrel 102 or remove the cartridge 130 from the barrel 102.

Once the cartridge 130 is loaded into the barrel 102, the user can pivot the barrel support 142 and the stock 104 relative to each other. The user can align the barrel support 142 and the stock 104 along the longitudinal axis 114. The pivot pin 188 moves along the curved portion of the groove 186b. The user can pivot the barrel support 142 and/or the stock 104 until the pivot pin 188 is aligned with the straight portion of the groove 186a. In some embodiments, the break trigger 192 is spring loaded. Once the pivot pin 188 is aligned with the straight portion of the groove 186a, the break trigger 192 moves the guide 184 distally under the action of the spring. The barrel support 142 and the stock 104 can be aligned along the longitudinal axis 114. The break trigger 192 can be distally positioned, ready for actuation by the user.

In some methods, the ratchet rod 136 is inserted through the lumen 162 of the base 160 of the stock 104 once the cartridge 130 is loaded. The delivery device 100 is in the first position as shown in FIGS. 1-5. The delivery device 100 can be actuated. As the trigger 166 is squeezed toward the handle 164, the trigger 166 interacts with the pawl 170 to cause the pawl 170 to rotate relative to the pivot pin 172. The pawl 170 advances the ratchet rod 136. The pawl 170 can advance along the gears 138. The ratchet rod 136 can push the material through the cartridge 130. The material can be pushed through the delivery opening 124 in the barrel 102. The material in the cartridge 130 is advanced with the interaction of the pawl 170 and ratchet rod 136. The rocker 176 limits or prevents the ratchet rod 136 from backing out.

In some methods of use, an additional cartridge 130 is needed to deliver more material to the surgical site. As the break trigger 192 is squeezed, the pivot pin 194 is guided by the break trigger 192 and moves proximally to move the guide 184. As the break trigger 192 is squeezed, the pivot pin 188 moves from the straight portion of the groove 186a to be aligned with the curved portion of the groove 186b to allow the pivoting motion of the delivery device 100. The ratchet rod 136 can be removed when the delivery device 100 is in the second position. The spent cartridge 130 can be removed when the delivery device 100 is in the second position. The ratchet rod 136 and the spent cartridge 130 can be removed together when the delivery device 100 is in the second position. The additional cartridge 130 can be loaded into the delivery device 100. The delivery device 100 can be pivoted to align the barrel support 142 with the stock 104. Once the pivot pin 188 is aligned with the straight portion of the groove 186a, the break trigger 192 is biased to move the guide 184 distally. In some methods, the ratchet rod 136 is inserted through the lumen 162 of the base 160 of the stock 104 after the delivery device 100 is pivoted into the first position. The break action 106 allows for easy loading. The break action 106 allows for easy reloading.

In some methods of use, the break action 106 is used before the delivery device 100 is positioned in the surgical site. In some methods of use, the cartridge 130 is loaded into the barrel 102 before the delivery device 100 is inserted into the surgical site. In some methods of use, the ratchet rod 136 is loaded into the barrel 102 before the delivery device 100 is inserted into the surgical site. In some methods of use, the ratchet rod 136 is loaded into the barrel 102 after the delivery device 100 is inserted into the surgical site. The break action 106 can be utilized before or after entry into the surgical site.

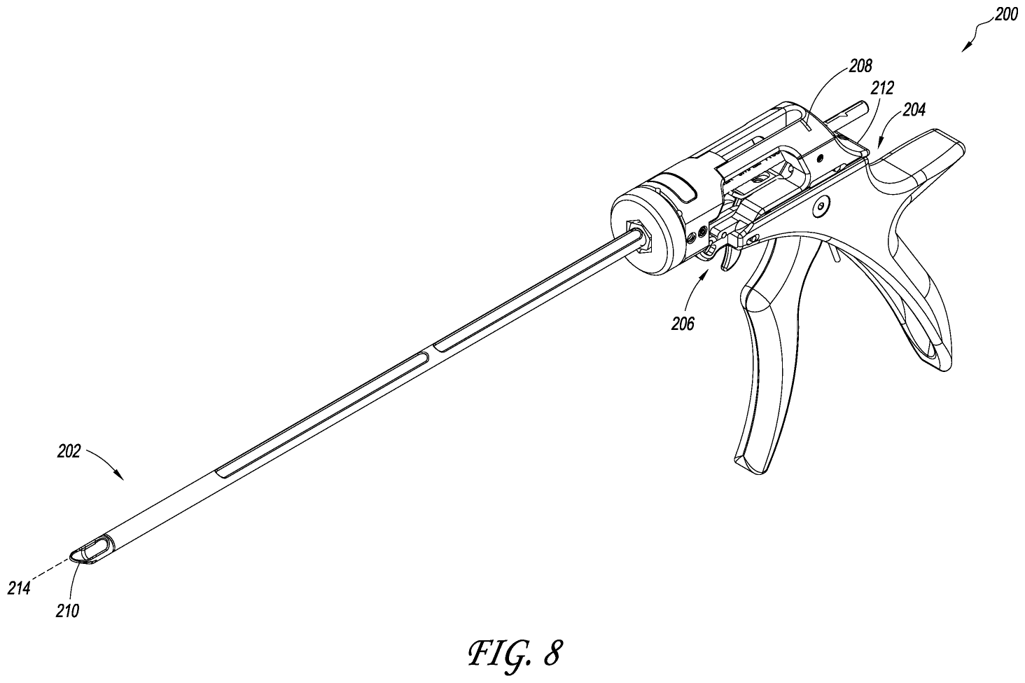

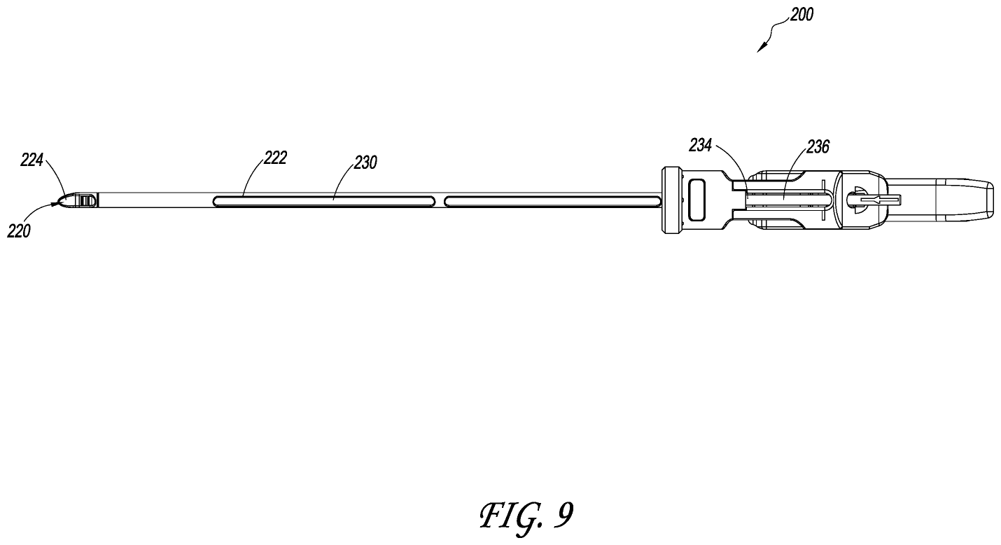

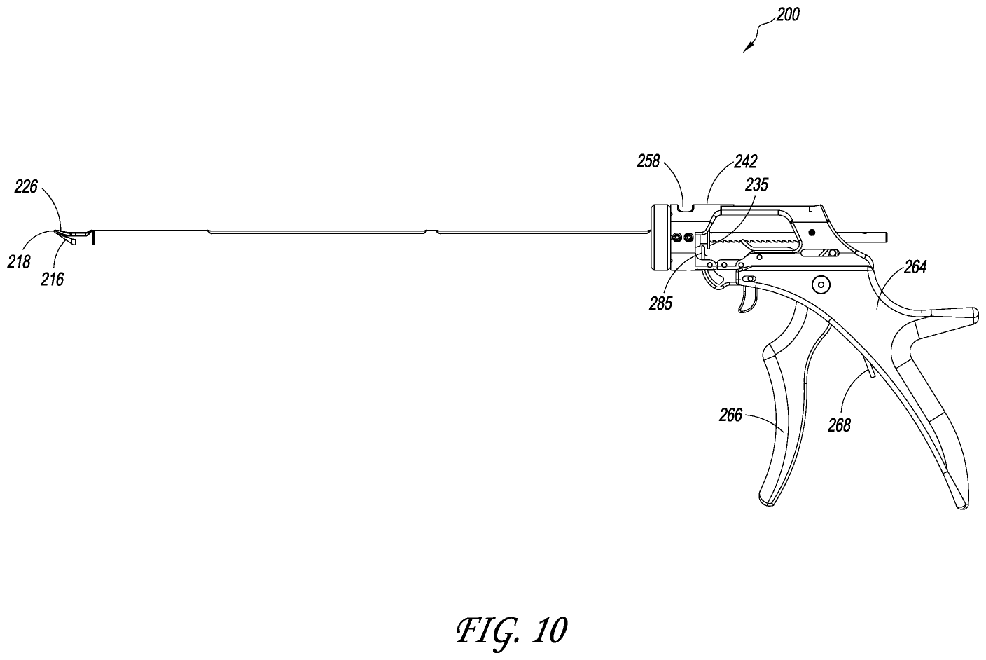

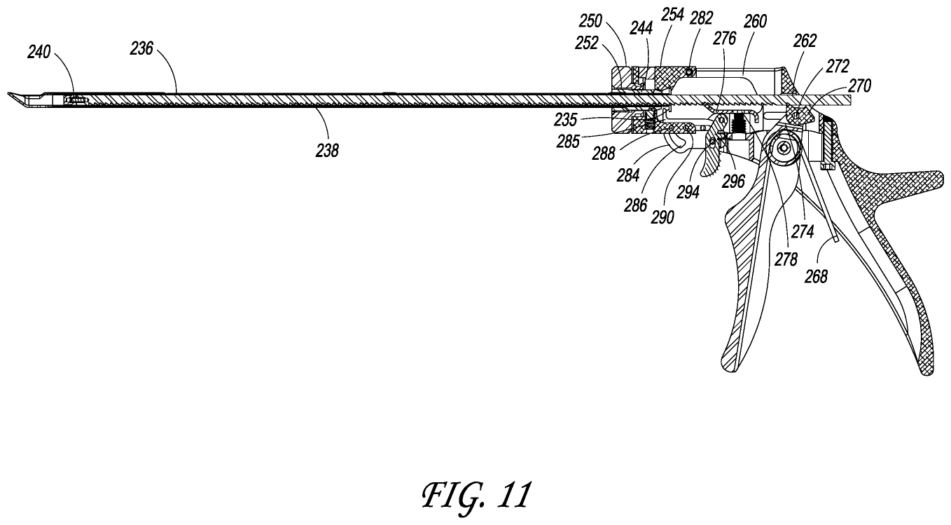

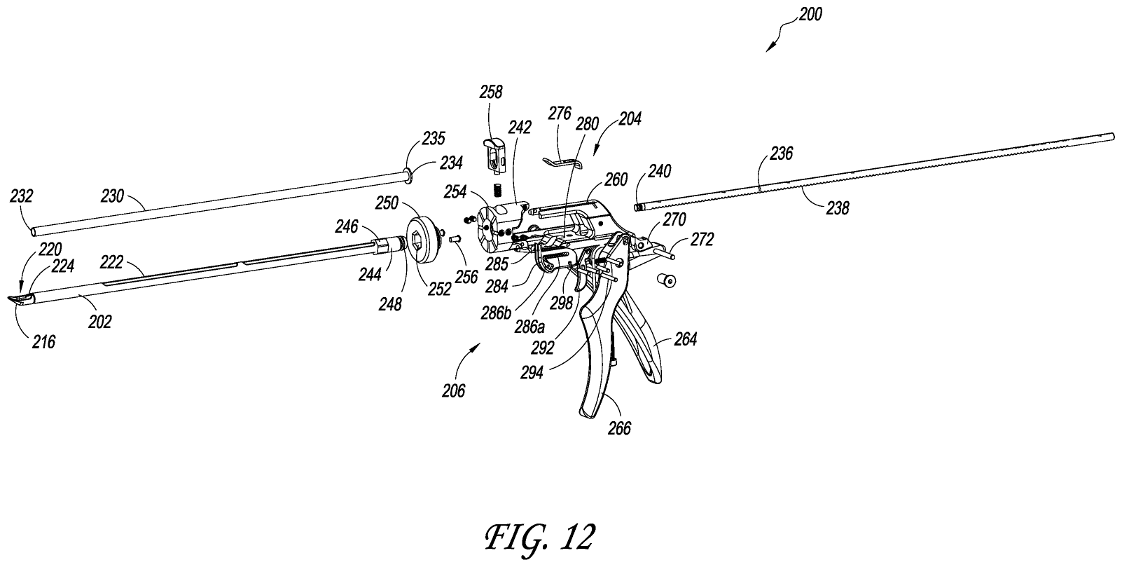

FIG. 8 shows a perspective view of an embodiment of a delivery device 200. FIG. 9 shows a top view of the delivery device 200. FIG. 10 shows a side view of the delivery device 200. FIG. 11 shows a cross-sectional view the delivery device 200. FIG. 12 is an exploded perspective view of the delivery device 200. The delivery device 200 can include any of the features of the delivery device 100 described herein.

As shown in FIGS. 8-12, the delivery device 200 can include similar features of delivery device 100. The delivery device 200 can include a barrel 202. The delivery device 200 can include a stock 204. The delivery device 200 can include a break action 206. The delivery device 200 can include a distal end 210 and a proximal end 212. The delivery device 200 can have a longitudinal axis 214 extending between the distal end 210 and the proximal end 212. The distal end 210 can include an insertion surface 216. The insertion surface 216 can include an atraumatic tip 218. The barrel 202 can include a lumen 220. The lumen 220 of the barrel 202 is sized to receive a cartridge 230. The barrel 202 can include one or more openings 222. The distal end 210 can include a delivery opening 224. The distal end 210 can include a lip 226. The proximal end of the barrel 202 can be coupled to a hub 244.

The delivery device 200 can include features to facilitate removal of the cartridge 230. The delivery device 200 can utilize the cartridge 230 comprising a flange 235. The cartridge 230 can form a tubular structure. The cartridge 230 can include a distal open end 232 and a proximal open end 234. The proximal open end 234 can include the flange 235. The flange 235 can be a circular flange. The flange 235 can be a semicircular flange. The flange 235 can have a larger cross-section or diameter than the tubular structure of the cartridge 230. The flange 235 can have any shape or configuration.

The delivery device 200 can include a ratchet rod 236. The cartridge 230 is sized to receive the ratchet rod 236 within the tubular structure of the cartridge 230. The ratchet rod 236 can include a series of gears 238. The ratchet rod 236 can include a plunger 240. The markings 208 can indicate the location for a measurement for the ratchet rod 236.

The delivery device 200 can include a barrel support 242. The barrel support 242 can include a rotation knob 250. The rotation knob 250 includes a keyed opening 252 sized to accept a keyed section 246 of the hub 244. The rotation knob 250 includes one or more ball plungers 256. The ball plungers 256 interact with a base 254 of the barrel support 242 to lock the rotational position of the barrel 202 relative to the barrel support 242. The barrel support 242 can include a button 258. The button 258 can allow the hub 244 to disengage from the rotation knob 250 to remove barrel 202.

The delivery device 200 can include the stock 204. The stock 204 can include a base 260. The base 260 of the stock 204 can include a lumen 262 sized to receive the ratchet rod 236. The stock 204 can include a handle 264. The handle 264 can be coupled to the base 260. The delivery device 200 can include a trigger 266. The trigger 266 can be actuated relative to the handle 264. The delivery device 200 can include a spring 268 to bias the trigger 266 outward from the handle 264.

The delivery device 200 can include a pawl 270. The pawl 270 can engage the ratchet rod 236 to advance the ratchet rod 236 relative to the cartridge 230. The delivery device 200 can include a pivot pin 272 extending through the trigger 266 and the pawl 270. The pawl 270 can be disposed within a groove 274 of the trigger 266. The pawl 270 can engage the ratchet rod 236 when the trigger 266 is actuated. The delivery device 200 can include a rocker 276. The rocker 276 can limit or prevent the proximal movement of the ratchet rod 236. The delivery device 200 can include a pivot pin 278 extending through the base 260 of the stock 204 and the rocker 276. The rocker 276 can engage the ratchet rod 236 by teetering along the ratchet rod 236 as the ratchet rod 236 moves. The rocker 276 can be disposed within a groove 280 of the base 260 of the stock 204.

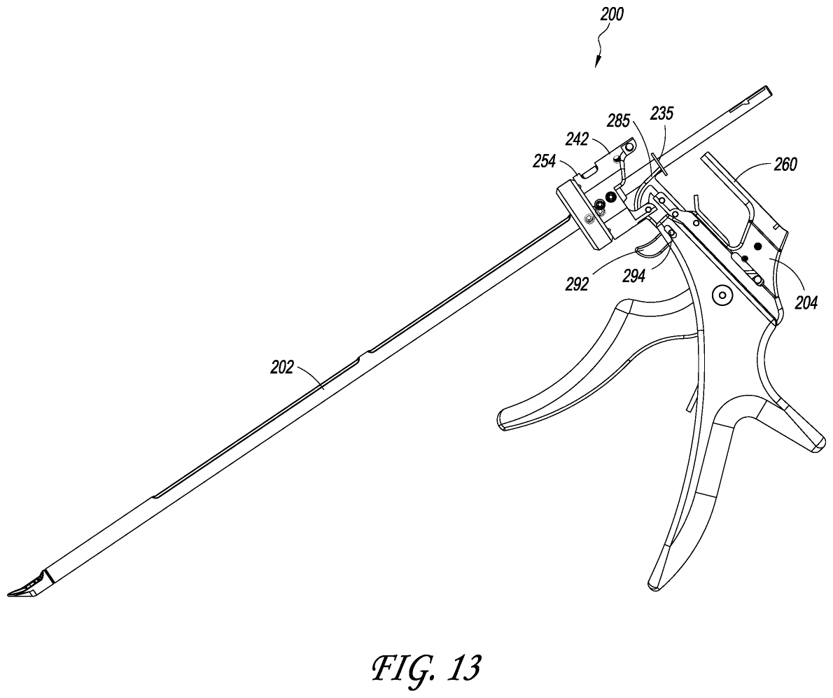

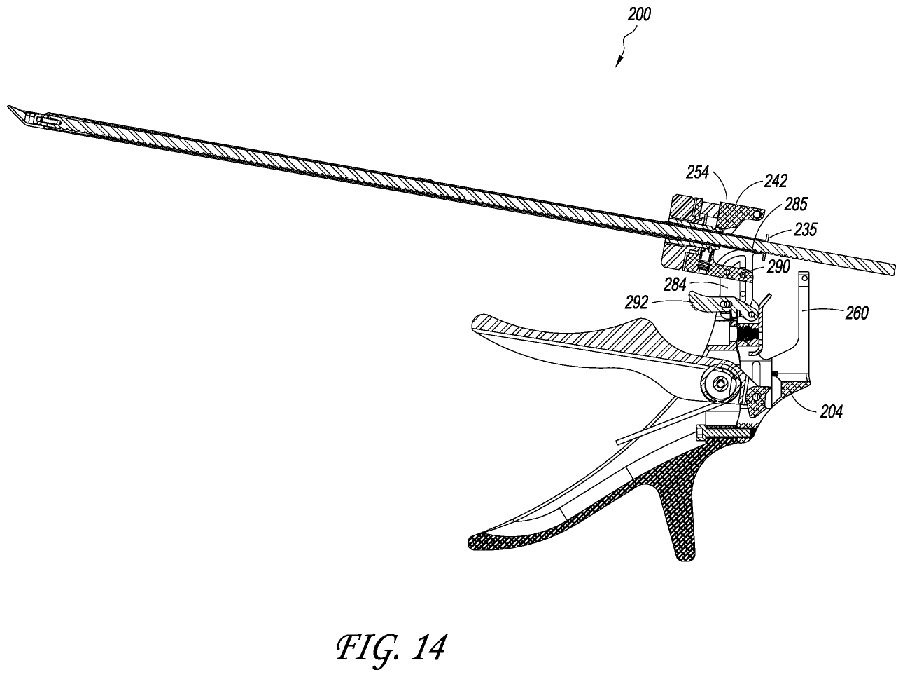

The delivery device 200 can include a break action 206. The break action 206 allows for loading of the cartridge 230 within the barrel 202. The device 200 can include features to facilitate the loading and the unloading of the cartridge 230. The features can be coupled with the break action 206. The break action 206 can include a pivot pin 282 disposed between the base 254 of the barrel support 242 and the base 260 of the stock 204. The barrel support 242 and the stock 204 can pivot about the pivot pin 282 in an upward and downward motion. The break action 206 can pivot about a horizontal axis. FIG. 13 is a perspective view of the delivery device 200 with the break action 206 activated. FIG. 14 is a cross-sectional view of the delivery device 200 with the break action 206 activated.

The break action 206 can include a guide 284. The guide 284 can include one or more ejector flanges 285. The one or more ejector flanges 285 can be prongs of the guide 284. The one or more ejector flanges 285 can extend upward from the guide 284. The one or more ejector flanges 285 can extend on either side of the cartridge 230. The ejector flanges 285 interact with the flange 235 of the cartridge 230. The ejector flanges 285 support the cartridge 230 upon activation of the break action 206. The ejector flanges 285 reliably position the cartridge 230 for removal by the user. When the break action 206 is released by actuating a break trigger 292, the one or more ejector flanges 285 catch the flange 235 of the cartridge 230. As the front half of the delivery device 200 pivots open along a curved portion of the groove 286b, the one or more ejector flanges 285 pull the cartridge 230 out of the barrel 202 to allow the user to easily remove the cartridge 230.

The guide 284 can include a groove 286. The groove 286 can include a straight portion 286a and the curved portion 286b. The delivery device 200 can include a pivot pin 288. The pivot pin 288 can extend through the base 254 of the barrel support 242 and within the groove 286 of the guide 284. When the pivot pin 288 is disposed within the straight portion of the groove 286a, the delivery device 200 cannot pivot. When the pivot pin 188 is disposed within the curved portion of the groove 286b, the delivery device 200 can pivot. Additional pins 290 can extend through the base 260 of the stock 204 and the straight portion of the groove 286a. The pivot pin 288 can be distal to the additional pins 290.

The break action 206 can include the break trigger 292. The break trigger 292 can be actuated by the index finger of the user. The break trigger 292 can be actuated while additional fingers of the user remain on the trigger 266. The delivery device 200 can include a pivot pin 294 disposed within a groove 296 of the break trigger 292 and disposed within a secondary groove 298 of the guide 284.

As the break trigger 292 is squeezed, the pivot pin 294 moves proximally. The guide 284 moves proximally as the break trigger 292 is squeezed. The one or more ejector flanges 285 move proximally. In some methods of use, the one or more ejector flanges 285 move the cartridge 230 proximally by moving the flange 235 of the cartridge 230 proximally.

As the break trigger 292 is squeezed and the guide 284 moves proximally, the pivot pins 290 slide along the straight portion of the groove 286a. The pivot pin 288 slides from the straight portion of the groove 286a to be aligned with the curved portion of the groove 286b. In this position of the pivot pin 288, the user can rotate the barrel support 242 downward such that the pivot pin 288 moves along the curved portion of the groove 288b. The user can rotate the barrel support 242 about the pivot pin 282 disposed between the base 254 of the barrel support 242 and the base 260 of the stock 204. In some methods of use, the one or more ejector flanges 285 support the flange 235 of the cartridge 230 during pivoting. The one or more ejector flanges 285 can position the flange 235 for easy removal by the user.

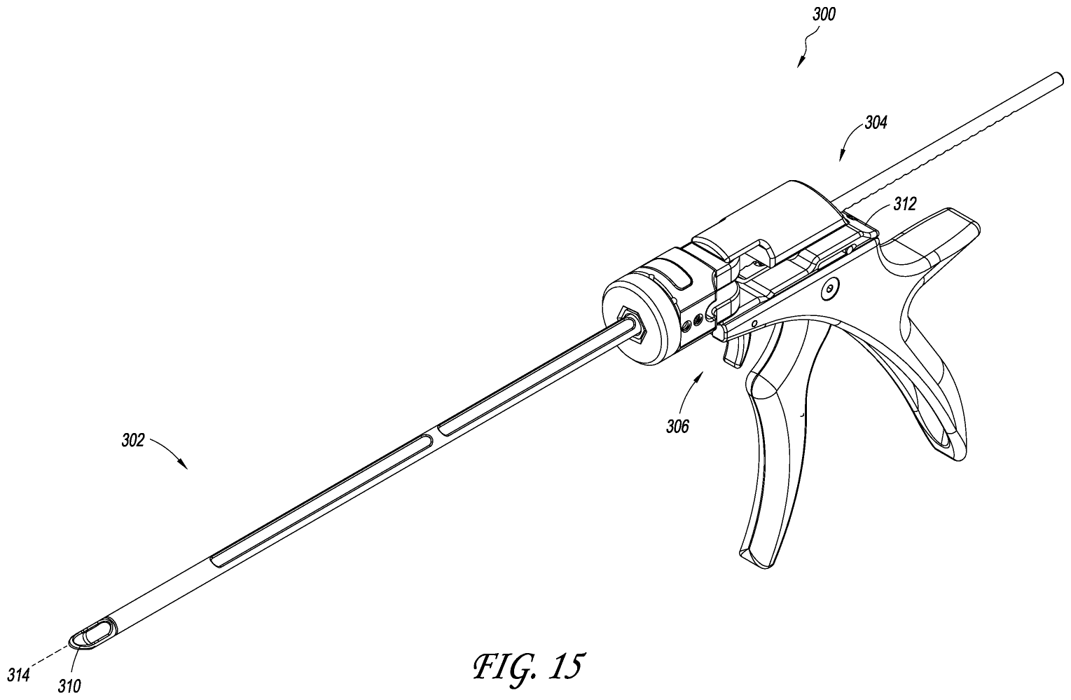

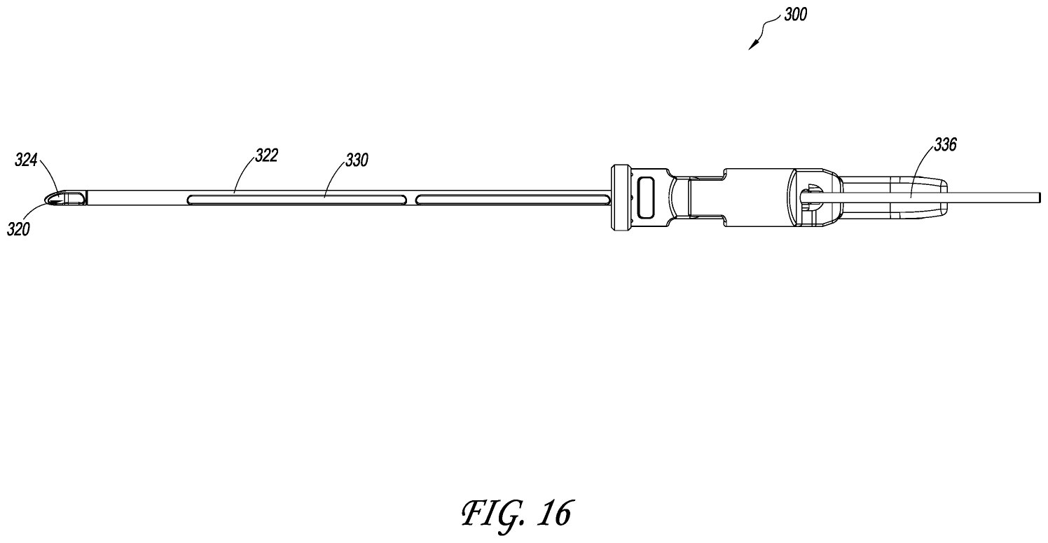

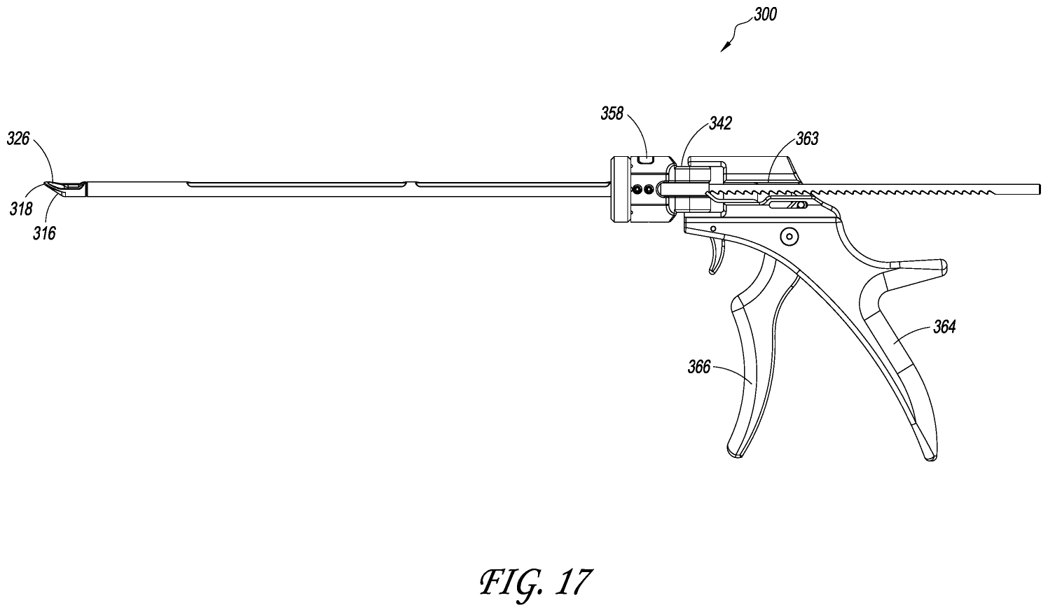

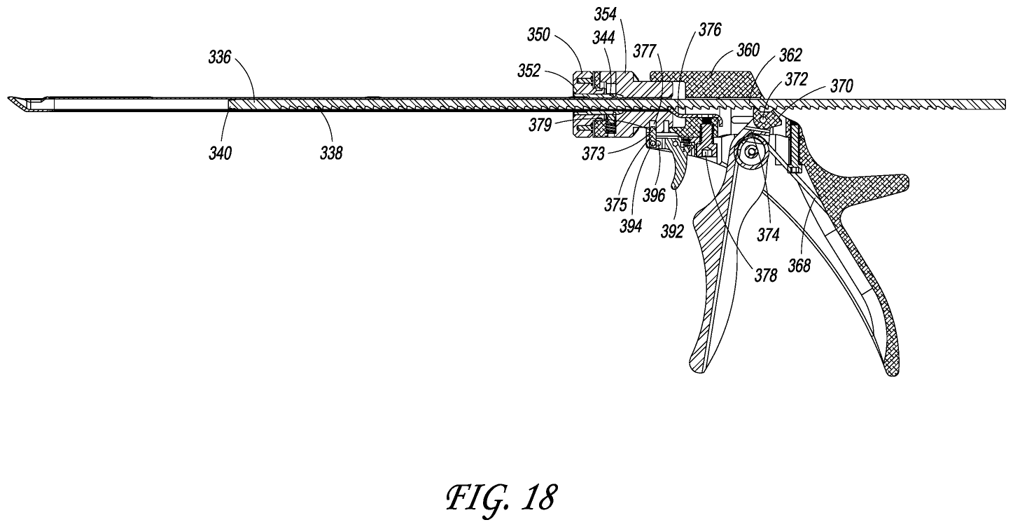

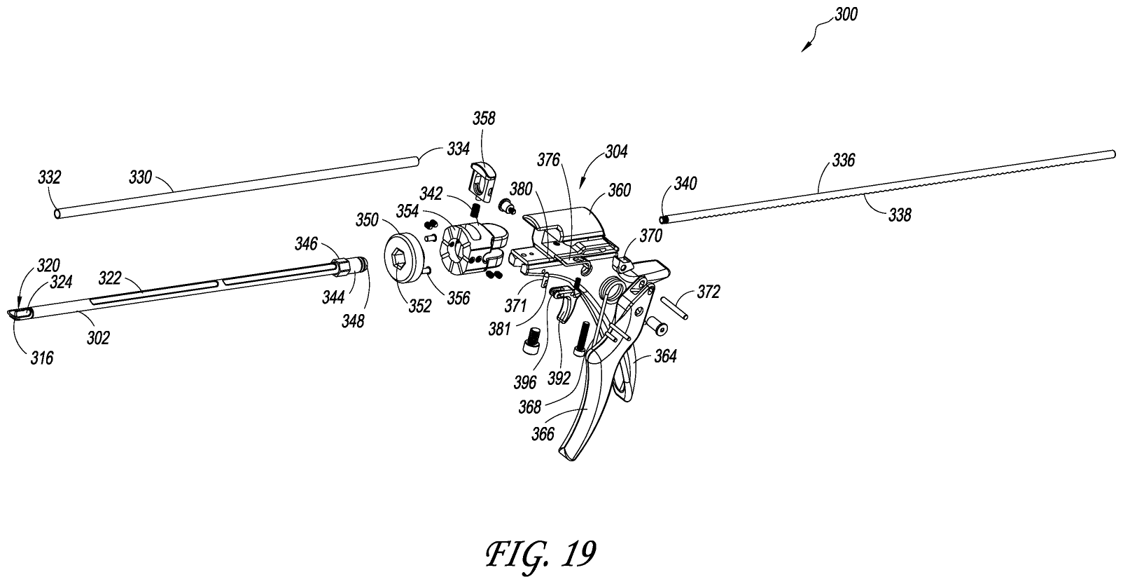

FIG. 15 shows a perspective view of an embodiment of a delivery device 300. FIG. 16 shows a top view of the delivery device 300. FIG. 17 shows a side view of the delivery device 300. FIG. 18 shows a cross-sectional view of the delivery device 300. FIG. 19 is an exploded perspective view of the delivery device 300. The delivery device 300 can include any of the features of the delivery device 100, 200 described herein. The delivery device 300 can pivot in a side to side motion.

As shown in FIGS. 15-19, the delivery device 300 can include a barrel 302. The delivery device 300 can include a stock 304. The delivery device 300 can include a break action 306. The break action 306 can break along a center line. The break action 306 can break along a side to side action instead of a downward motion. The delivery device 300 can include a different mechanism for the break action 306. The break action 306 can pivot about a vertical axis. The delivery device 300 can include different design features in the stock 304 to facilitate the side to side action. The delivery device 300 breaks out of plane. The delivery device 300 breaks in a side action.

The delivery device 300 can include a distal end 310 configured to be inserted in a patient and a proximal end 312 configured to be external to the patient. The delivery device 300 can have a longitudinal axis 314 extending between the distal end 310 and the proximal end 312. The barrel 302 can be coaxial with the longitudinal axis 314. The barrel 302 can include an insertion surface 316 configured for insertion into the body of the patient. The insertion surface 316 can include an atraumatic tip 318 to facilitate insertion. The barrel 302 can include a lumen 320. The barrel 302 can include one or more openings 322. The barrel 302 can include a delivery opening 324. The barrel 302 can include a lip 326 to facilitate the delivery of material.

The lumen 320 of the barrel 302 is sized to receive a cartridge 330. The cartridge 330 can be any size or shape and the lumen 320 can be any corresponding size or shape. The cartridge 330 can include a distal open end 332 and a proximal open end 334. The cartridge 330 is sized to receive a ratchet rod 336. The ratchet rod 336 can include a series of gears 338. The ratchet rod 336 can include a plunger 340 to push material from the cartridge 330.

The delivery device 300 can include a barrel support 342 configured to support the barrel 302. The proximal end of the barrel 302 can be coupled to a hub 344 including a keyed section 346 and a groove 348. The barrel support 342 can include a rotation knob 350 including a keyed opening 352. The keyed opening 352 can be sized to accept the keyed section 346 of the hub 344. Rotation of the rotation knob 350 can cause a corresponding rotation of the hub 344 and the barrel 302. The barrel support 342 can include a base 354. The rotation knob 350 can include one or more ball plungers 356 to lock the rotational position of the barrel 302 relative to the base 354. The barrel support 342 can include a button 358 to allow the hub 344 to disengage from the rotation knob 350 to remove barrel 302. The rotation knob 350 can be rotated to any position, similar to the rotation knobs 150, 250. The delivery opening 324 can be at any circumferential position, similar to the delivery openings 124, 224 of the delivery devices 100, 200.

The delivery device 300 can include the stock 304 including a base 360. The base 360 of the stock 304 can include a side slot 363. The side slot 363 is open to one side of the base 360. In the illustrated embodiment, the side slot 363 is open to the left side when the user is holding the delivery device 300 for use. The side slot 363 allows the cartridge 330 to extend along the longitudinal axis 314 when the delivery device 300 is pivoted. The side slot 363 allows the ratchet rod 336 to extend along the longitudinal axis 314 when the delivery device 300 is pivoted. The side slot 363 allows the delivery device 300 to pivot in a side to side motion. The side slot 363 allows the delivery device 300 to open up before ratchet rod 336 has fully advanced. This action to open up the delivery device 300 disengages a pawl 370 and the ratchet rod 336 can be removed.

The stock 304 can include a handle 364. The handle 364 can be coupled to the base 360. The delivery device 300 can include a trigger 366. The trigger 366 can be actuated to move the ratchet rod 336. The delivery device 300 can include a spring 368 to bias the trigger 366 relative to the handle 364.

The delivery device 300 can include the pawl 370 disposed about a pivot pin 372. The pivot pin 372 can extend through the trigger 366 such that the pawl 370 is disposed within a groove 374 of the trigger 366. The trigger 366 can be actuated to move the ratchet rod 336 by advancing the pawl 370. The delivery device 300 can include a rocker 376 disposed about a pivot pin 378. The pivot pin 378 can extend through the base 360 of the stock 304 and the rocker 376. The rocker 376 is a passive stop which can limit or prevent the proximal movement of the ratchet rod 336. The rocker 376 can be disposed within a groove 380 of the base 360 of the stock 304.

The delivery device 300 can include the break action 306. The break action 306 allows for loading and/or unloading of the cartridge 330 within the barrel 302. The break action 306 opens the delivery device 300 in a side to side motion.

The break action 306 can include a pin 371. The pin 371 can be disposed between the base 354 of the barrel support 342 and the base 360 of the stock 304. The base 354 of the barrel support 342 and the base 360 of the stock 304 overlap with the pin 371 disposed through both. The pin 371 can be vertical to allow for the side to side motion. The pin 371 can include a tapered tip 373. The pin 371 can include an interference portion 375. The base 354 of the barrel support 342 can include a lumen 377. The lumen 377 can be sized to receive the tapered tip 373. The lumen 377 can be sized to form a friction fit with the interference portion 375. When the interference portion 375 and the tapered tip 373 is disposed within the lumen 377 of the base 354 of the barrel support 342, the delivery device 300 cannot pivot. When pin 371 moves downward within the lumen 377 of the base 354 of the barrel support 342, the delivery device 300 can pivot. As long as the tapered tip 373 is removed from barrel support 342, the stock 304 can pivot. The interference portion 375 of the pin 371 can extend through a lumen 379 in the base 360 of the stock 304. The lumen 379 can be sized to form a friction fit with the interference portion 375. The base 360 of the stock 304 cannot rotate relative to the pin 371. The pivot of the delivery device 300 can be the pin 371. The pivot is centered over the central line of the barrel 302. The pivot is centered over the central line of the cartridge 330. The pivot is centered over the central line of the barrel support 342. The pivot is centered over the central line of the body of the delivery device. The pivot is centered over the longitudinal axis 314.

The break action 306 can include a break trigger 392. The delivery device 300 can include a pivot pin 394. The pivot pin 394 is disposed within a groove 396 of the break trigger 392. The pivot pin 394 is disposed within a groove 381 of the pin 371. The pin 371 is configured to move in a vertical direction in response to the break trigger 392.

As the break trigger 392 is squeezed, the pin 371 moves downward. The pin 394 moves downward as the break trigger 392 is squeezed. The break action is released by pulling the break trigger 392. The break trigger 392 pulls the pin 371 out of the barrel support 342. The pin 371 can be spring loaded. The pin 371 can be biased into engagement with the barrel support 342. The break trigger 392 can overcome this biasing force. The barrel support 342 is a rotating front body portion of the delivery device 300.

As the break trigger 392 is squeezed and the pin 371 moves downward, the interference portion 375 slides out of engagement with the lumen 377 of the base 354 of the barrel support 342. The tapered tip 373 slides into the lumen 379 of the base 360 of the stock 304. The user can apply a rotational force to the stock 304. The pin 371 allows rotation between the barrel support 342 and the stock 304. The cartridge 330 and the ratchet rod 336 can extend from the side slot 363 when the barrel support 342 is rotated relative to the stock 304. The user can rotate the stock 304 counter clockwise when the delivery device 300 is viewed from the top to load or unload the cartridge. The user can rotate the stock 304 clockwise to align with the loaded cartridge 330 and the longitudinal axis 314. The user can rotate the stock 304 to load or unload the cartridge. The user can rotate the stock 304 once the cartridge is loaded. Once the stock 304 and the barrel support are aligned 342, the user can actuate the trigger 366 to advance the ratchet rod 326 relative to the cartridge 330.

FIGS. 15-19 are views of the delivery device 300 in a first position. The delivery device 300 can be inserted into the surgical site in this position. The delivery device 300 can advance material to the surgical site in this position. In the first position, the base 354 of the barrel support 342 is generally aligned with the base 360 of the stock 304 along the longitudinal axis 314. The barrel 302 is coupled with the barrel support 342 through the connection of the hub 344 and the rotational knob 350. The barrel 302 can be rotated relative to the barrel support 342 to position the delivery opening 324. The barrel 302 is generally coaxial with rest of the delivery device 300. The interference portion 375 is disposed within the lumen 377 of the base 354 of the barrel support 342. The delivery device 300 cannot rotate. In some methods of use, the delivery device 300 can be impacted or driven into the surgical site.

In some methods of use, the cartridge 330 and the ratchet rod 336 are not loaded into the barrel 302 when the delivery device 300 is being inserted into the surgical site. In some methods of use, the cartridge 330 and the ratchet rod 336 are loaded once the barrel 302 is in position at the surgical site. In some methods of use, the break action 306 can be utilized after entry into the surgical site.

The delivery device 300 can include the break trigger 392. As the break trigger 392 is squeezed, the pin 371 moves downward and out of the interference with the lumen 377 of the base 354 of the barrel support 342. Once the pin 371 moves downward, the user can rotate the stock 304 relative to the barrel support 342. Once the pin 371 moves downward, the user can rotate the barrel support 342 relative to the stock 304. The delivery device 300 opens through a side to side motion. The delivery device 300 has a break action function. The break action 306 allows the delivery device 300 to pivot relative to the barrel 302 that is located at the surgical site.

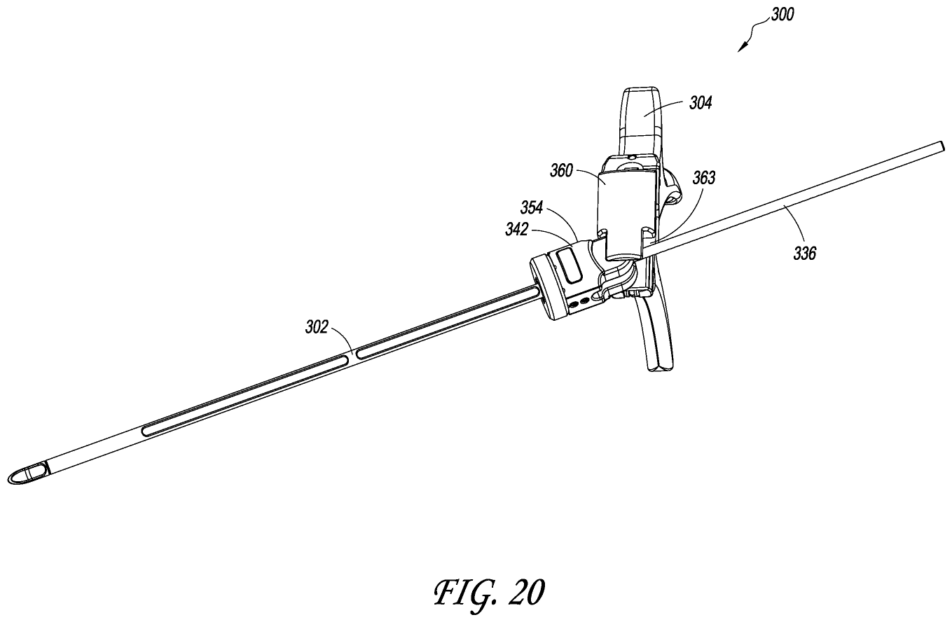

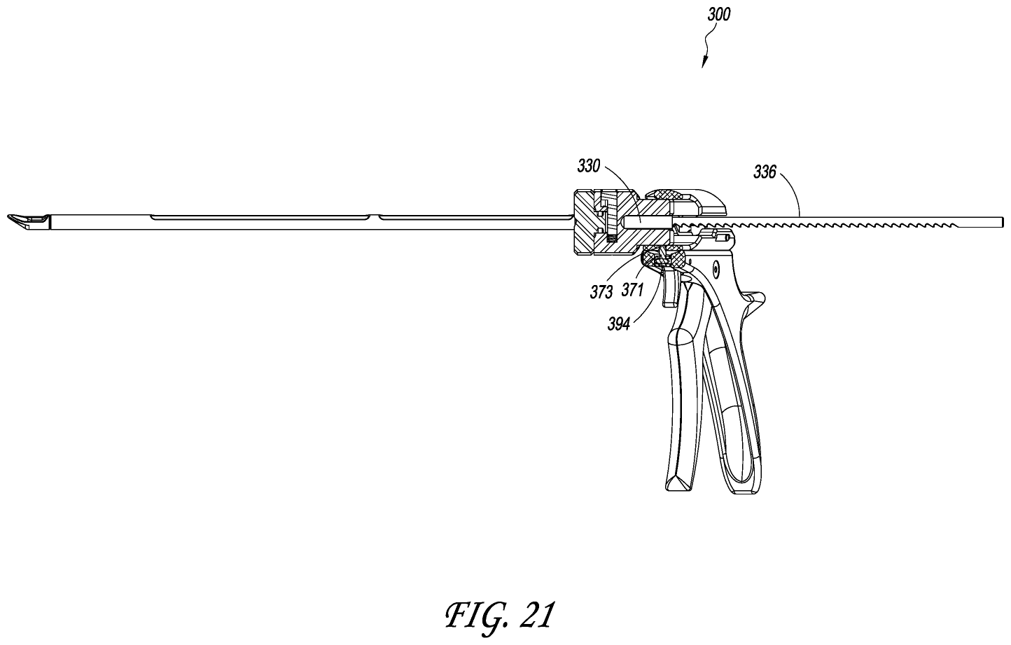

FIGS. 20-21 are views of the delivery device 300 in a second position. FIG. 20 is a perspective view of the delivery device 300 with the break action 306 activated. FIG. 21 is a cross-sectional view of the delivery device 300 with the break action 306 activated. Once the delivery device 300 is pivoted, the cartridge 330 can be easily loaded into the barrel 302. The cartridge 330 can be slid into the barrel 302. The user can visualize the opening of the barrel 302 when the delivery device 300 is pivoted. Once the cartridge 330 is loaded, the user can rotate the stock 304 to be aligned with the barrel 302. Once the cartridge 330 is loaded, the user can rotate the stock 304 and the barrel support 342 relative to each other. In some embodiments, the break trigger 392 is spring loaded. The break trigger 392 can move the pin 371 upward under the action of the spring.

In some methods, the ratchet rod 336 is inserted through the lumen 362 of the base 360 of the stock 304. The delivery device 300 can be actuated. As the trigger 366 is squeezed toward the handle 364, the trigger 366 interacts with the pawl 370 to advance the ratchet rod 336. The ratchet rod 336 can advance the material through the cartridge 330 and through the delivery opening 324. In some methods of use, the rocker 376 limits or prevent the ratchet rod 336 from advancing proximally.

In some methods of use, an additional cartridge 330 is needed. As the break trigger 392 is squeezed, the pin 371 moves downward. The user can rotate the stock 304 and/or the barrel support 342. The spent cartridge 330 and the ratchet rod 336 can be removed when the delivery device 300 is in the second position. The additional cartridge 330 can be loaded into the delivery device 300. The stock 304 and the barrel support 362 can be rotated into alignment along the longitudinal axis 314. The break trigger 392 can move the pin 371 upward under the action of the spring. In some methods, the ratchet rod 336 is inserted through the lumen 362 of the base 360 of the stock 304 after the delivery device 300 is pivoted. The break action 306 allows for easy loading and unloading of the cartridge 330.

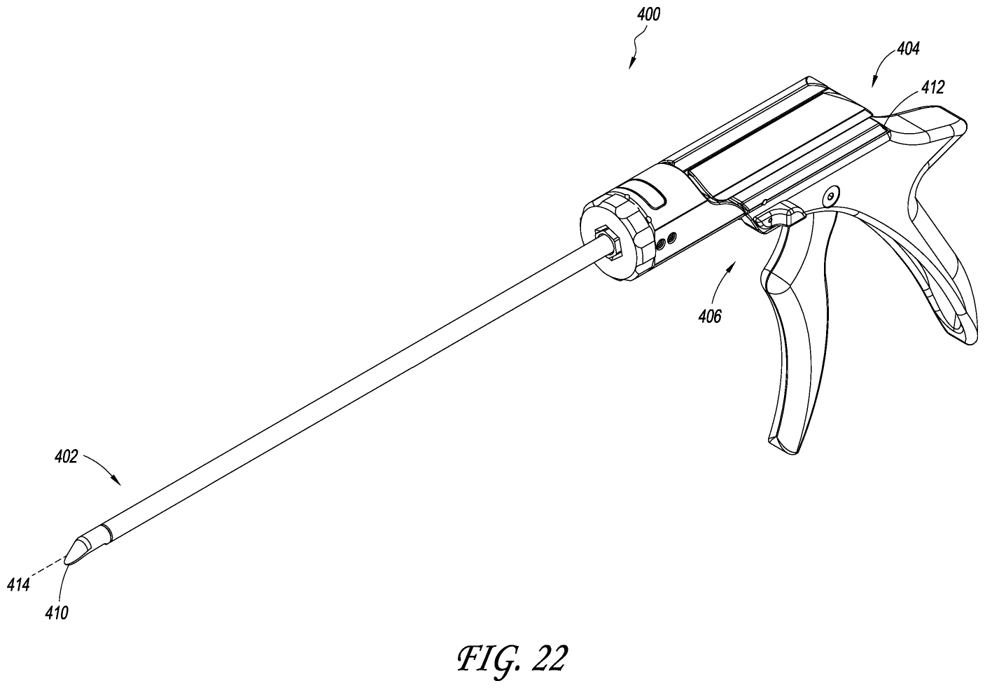

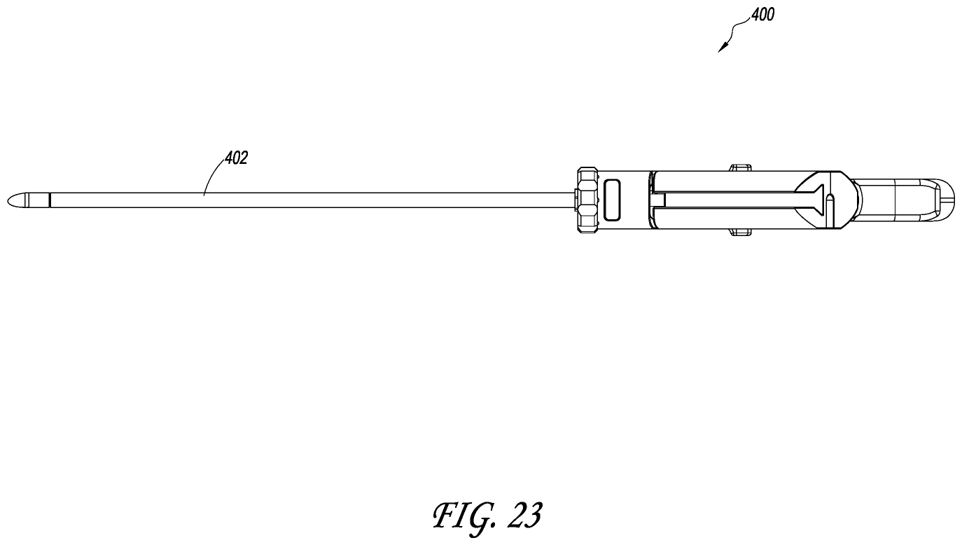

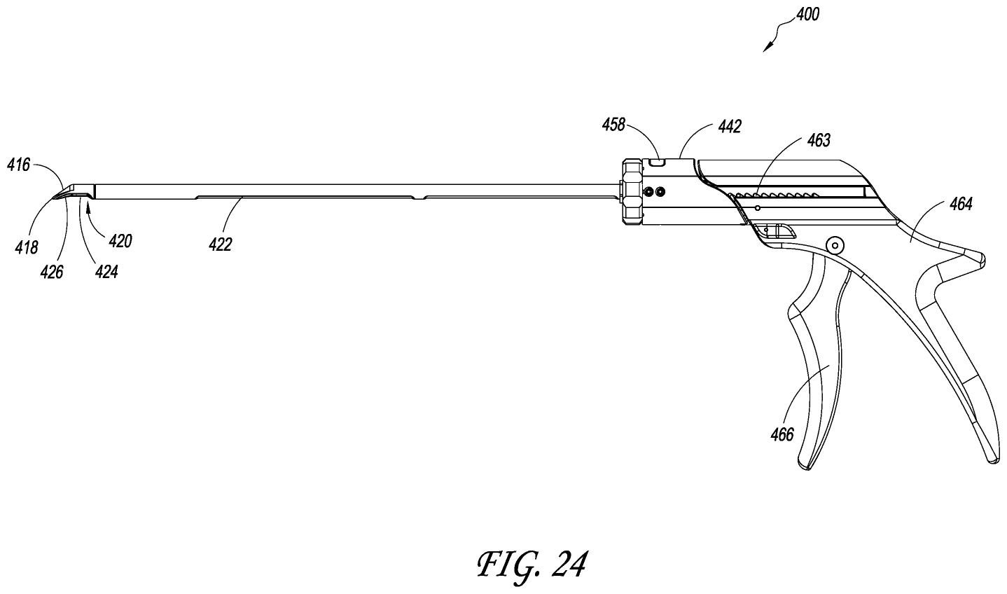

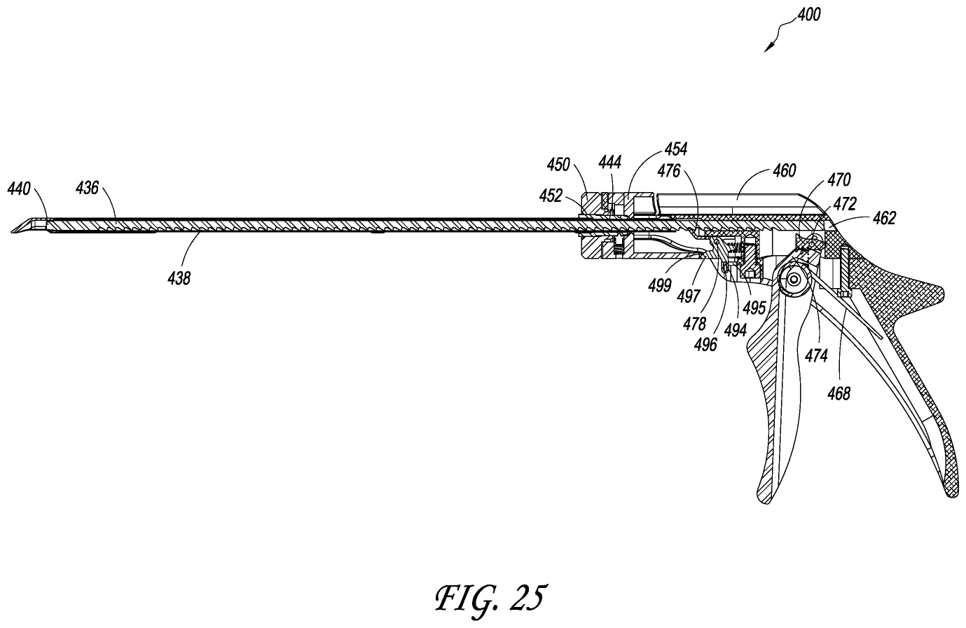

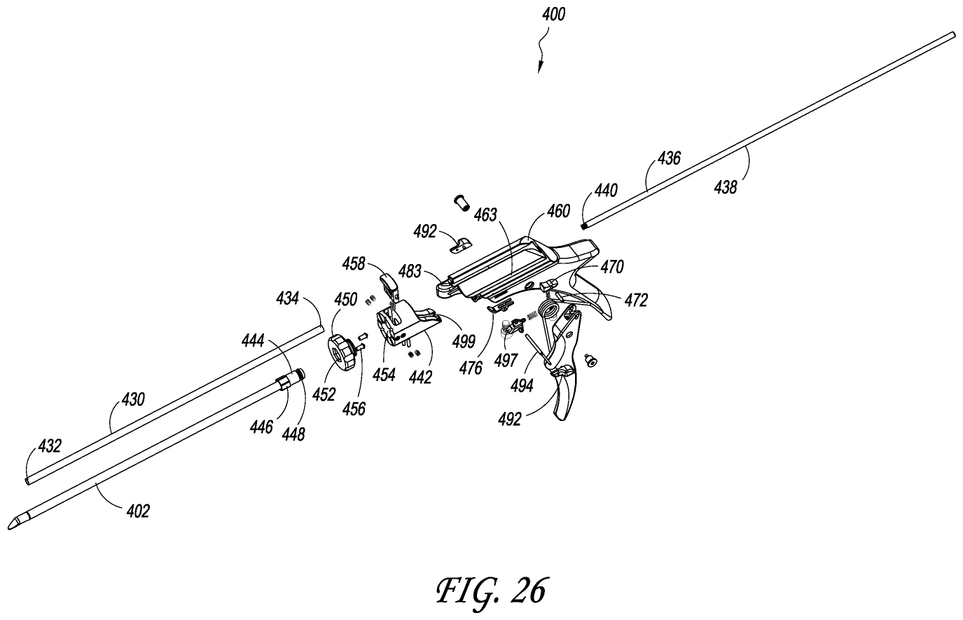

FIG. 22 shows a perspective view of an embodiment of a delivery device 400. FIG. 23 shows a top view of the delivery device 400. FIG. 24 shows a side view of the delivery device 400. FIG. 25 shows a cross-sectional view of the delivery device 400. FIG. 26 is an exploded perspective view of the delivery device 400. The delivery device 400 can include any of the features of the delivery device 100, 200, 300 described herein.

As shown in FIGS. 22-26, the delivery device 400 can include a barrel 402. The delivery device 400 can include a stock 404. The delivery device 400 can include a break action 406. The break action 406 can pivot in a side to side action. The delivery device 400 can include a different mechanism for the break action 406 than the break action 306. The break action 406 can pivot about a vertical axis. The delivery device 400 can include design features in the stock 404 to facilitate the side to side action. The delivery device 400 breaks out of plane. The delivery device 400 breaks in a side action.

The delivery device 400 can include a distal end 410 and a proximal end 412. The delivery device 400 can include a longitudinal axis 414. The break action 406 can be perpendicular to the longitudinal axis 414. The distal end 410 can include an insertion surface 416. The insertion surface 416 can include an atraumatic tip 418. The distal end 410 can include any feature to facilitate insertion of the barrel 402 to the surgical site. The distal end 410 can include any feature to facilitate directed delivery of material to the surgical site.

The barrel 402 can include a lumen 420. The lumen 420 of the barrel 402 is sized to receive a cartridge 430. The barrel 402 can include one or more openings 422. In some embodiments, the openings 422 allow the user to view the cartridge 430 and/or the ratchet rod 436. The distal end 410 can include a delivery opening 424. The delivery opening 424 can be oriented at one of a plurality of positions prior to insertion into the patient. The distal end 410 can include a lip 426 to direct the flow of material. In some embodiments, the proximal end of the barrel 402 is coupled to a hub 444. The hub 444 can include a keyed section 446 and a groove 448.

The delivery device 400 is sized to receive a cartridge 430. The cartridge 430 can form a tubular structure containing material to be delivered. The cartridge 430 can include a distal open end 432 and a proximal open end 434. The cartridge 430 delivers material through the distal open end 432. The cartridge 430 is sized to receive a ratchet rod 436 through the proximal open end. The ratchet rod 436 can include a series of gears 438 configured to be engaged to advance the ratchet rod 436 relative to the cartridge 430. The ratchet rod 436 can include a plunger 440.

The delivery device 400 can include a barrel support 442. The barrel support 442 can include a rotation knob 450. The rotation knob 450 includes a keyed opening 452 shaped to accept the keyed section 446 of the hub 444. Rotation of the rotation knob 450 causes rotation of the hub 444 and the barrel 402. The barrel support 442 can include a base 454. The rotation knob 450 includes one or more ball plungers 456 or other feature to lock the rotational position of the barrel 402 relative to the barrel support 442. The barrel support 442 can include a button 458 or other feature to allow removal of barrel 402. The rotation knob 450 can be rotated to any position, similar to the rotation knobs 150, 250, 350. The delivery opening 424 can be any circumferential position, similar to the delivery opening 124, 224, 324 of the delivery devices 100, 200, 300.

The delivery device 400 can include the stock 404. The stock 404 can include a base 460. The base 460 of the stock 404 can include a side slot 463. The side slot 463 is open to the side of the base 460. The base 460 is closed around a substantial portion of the ratchet rod 436 and/or cartridge 430. The base 460 is closed around 40% of the circumference, 45% of the circumference, 50% of the circumference, 55% of the circumference, 60% of the circumference, 65% of the circumference, 70% of the circumference, 75% of the circumference, 80% of the circumference, 90% of the circumference, or any range of the foregoing values. The side slot 463 is open to the side of the base 460 to allow the ratchet rod 436 and/or cartridge 430 to slide through the side slot 463. In the illustrated embodiment, the side slot 463 is open on the left side when the user is holding the delivery device 400 for use. The stock 404 can include a handle 464. The delivery device 400 can include a trigger 466. The delivery device 400 can include a spring 468 to bias the trigger 466.

The delivery device 400 can include a pawl 470. The pawl 470 can have a smaller, compact shape than the pawl 170, 270, 370. Any size or shape pawl is contemplated that functions to engage the ratchet rod 436. The delivery device 400 can include a pivot pin 472. The pivot pin 472 can extend through the trigger 466 and the pawl 470. The pawl 470 can be disposed within a groove 474 of the trigger 466. The delivery device 400 can include a rocker 476. The delivery device 400 can include a pivot pin 478. The pivot pin 478 can extend through the base 460 of the stock 404 and the rocker 476. The rocker 476 can have a smaller, compact shape than the rocker 176, 276, 376. Any size or shape rocker is contemplated that functions to engage the ratchet rod 436.

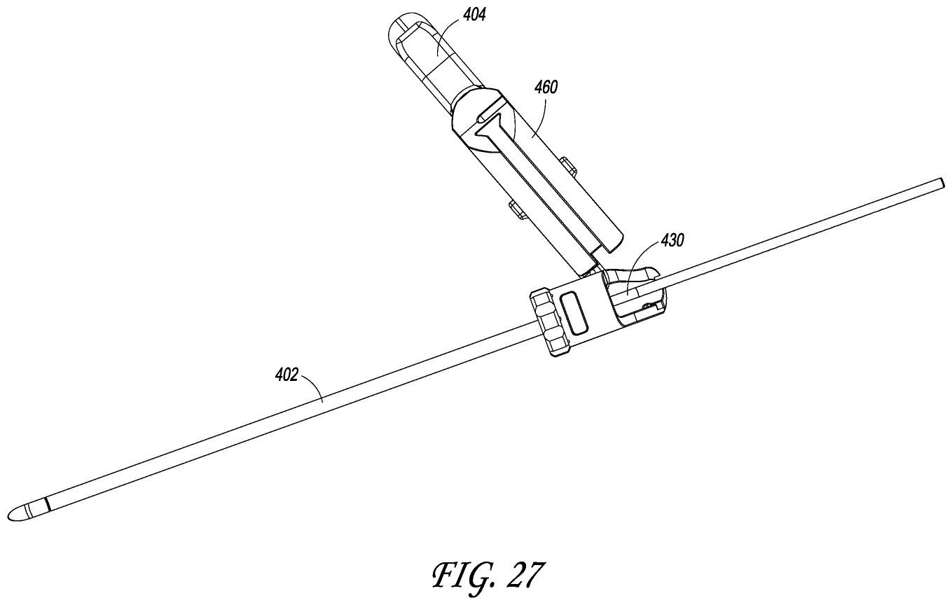

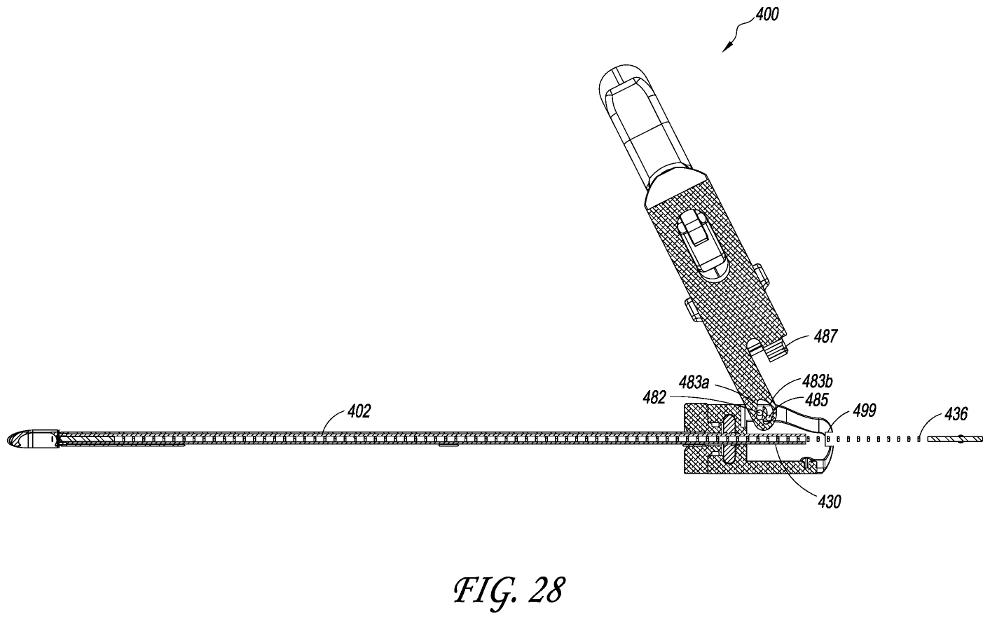

The delivery device 400 can include the break action 406. The break action 406 allows for loading and unloading the cartridge 430 within the barrel 402. The break action 406 can have a different shaped trigger. The break action 406 can have a compact design. The break action 406 can allow for side to side motion of the base 454 of the barrel support 442 relative to the base 460 of the stock 404. The break action 406 can include a pivot pin 482. The pivot pin 482 can be disposed between the base 454 of the barrel support 442 and the base 460 of the stock 404. The pivot pin 482 can be disposed perpendicular to the longitudinal axis 414. The pivot pin 482 can be disposed to allow the base 454 of the barrel support 442 to pivot left or right relative to the base 460 of the stock 404. The pivot pin 482 can be disposed to allow the base 460 of the stock 404 to pivot left or right relative to the base 454 of the barrel support 442. FIG. 27 is a perspective view of the delivery device 400 with the break action 406 activated. FIG. 28 is a cross-sectional view of the delivery device 400 with the break action 406 activated.

The pivot of the delivery device 400 can include the pivot pin 482. The pivot is off centered from the central line of the barrel 402. The pivot is off centered from the central line of the cartridge 430. The pivot is off centered from the central line of the barrel support 442. The pivot is off centered from the central line of the body of the delivery device 400. The pivot is off centered from the longitudinal axis 414.

The base 460 of the stock 404 can include a groove 483. The groove 483 can include a straight portion 483a. The straight portion 483a can extend through the base 460 of the stock 404. The pivot pin 482 can extend through the base 454 of the barrel support 442. The pivot pin 482 can extend within the straight portion 483a. The groove 483 can include a curved portion 483b. The straight portion 483a can extend through a portion of the base 460 of the stock 404. The delivery device 400 can include a secondary pivot pin 485. The secondary pivot pin 485 can extend upward from the base 454 of the barrel support 442. The secondary pivot pin 485 can extend within the curved portion 483b. The secondary pivot pin 485 can function as a cam.

The pivot of the delivery device 400 can include a slanted slot or the straight portion 483a. The pivot of the delivery device 400 can include a cammed slot or the curved portion 483b. The pivot of the delivery device 400 includes the slanted slot and cam. The straight portion 483a allows the two halves of the delivery device 400 to pull together when under axial load. The straight portion 483a and the pivot pin 482 allow the barrel support 442 and the stock 406 to come together. The straight portion 483a and the pivot pin 482 allow the barrel support 442 and the stock 406 to compress toward each other under axial loading. The curved portion 483b and the secondary pivot pin 485 can allow the barrel support 442 and the stock 406 to pivot.

The pivot of the delivery device 400 can include a guide 487. This axial loading engages the guide 487 on the opposite side of the delivery device 400 as the pivot. The guide 487 can prevent the delivery device 400 from opening up due to the off center pivot. The guide 487 can be disposed between the base 454 of the barrel support 442 and the base 460 of the stock 404. The guide 487 can include a protrusion on the base 460 of the stock 404 and a corresponding recess on the base 454 of the barrel support 442. In other embodiments, the guide 487 can include a protrusion on the base 454 of the barrel support 442 and a corresponding recess on the base 460 of the stock 404. In the illustrated embodiment, the protrusion includes a T-shaped or dog-bone configuration. Other shapes of protrusions are contemplated. The guide 487 can be located opposite the off center pivot. The guide 487 engages the two body halves of the delivery device 400 in axial load. The protrusion on one half of the body engages the recess in the second half of the body, on the side opposite the off center pivot. The guide 487 can take up the axial load and prevent the pivot from opening.

The break action 406 can include a break trigger 492. The break trigger 492 can be slide mechanism. The break trigger 492 can be actuated by the index finger of the user while the remaining fingers wrap around the trigger 466. The break trigger 492 can include a dual slide. The break trigger 492 can include a slide on the right side of the delivery device 400. The break trigger 492 can include a slide on the left side of the delivery device 400. The break trigger 492 can include a slide on both the right side and the left side of the delivery device 400 as illustrated. The break trigger 492 can include a slide on both the right side and the left side of the delivery device 400 to allow ambidextrous use.

In some embodiments, the rocker 476 is coupled to the break trigger 492. The rocker 476 can be coupled to the break trigger 492 for a compact design of the delivery device 400. The rocker 476 can slide when the break trigger 492 slides. The rocker 476 can teeter along the ratchet rod 436 from any position of the break trigger 492. In other embodiments, the rocker 476 and the break trigger 492 are not coupled.

The delivery device 400 can include a pivot pin 494 to allow the break trigger 492 to slide relative to the handle 464. The pivot pin 494 is disposed within a groove 496 of the break trigger 492. The pivot pin 494 is disposed within a groove 495 in the handle 464. The break action 406 can include a stop 497. In some embodiments, the stop 497 can be integrally formed in the break trigger 492. The stop 497 abuts a cutout 499 in the base 454 of the barrel support 442 when the break trigger 492 is not depressed. When the stop 497 abuts the cutout 499 in the base 454 of the barrel support 442, the base 454 of the barrel support 442 is prevented or limited from pivoting left or right relative to the base 460 of the stock 404. The break action is initiated by pulling the break trigger 492 with the index finger of the user. The break trigger 492 pulls stop 497 out of the cutout 499 of the barrel support 442. The stop 497 can be spring loaded. The stop 497 can be biased into engagement with the barrel support 442. The barrel support 442 can be the front half of the delivery device. Pulling the stop 497 from the cutout 499 allows the barrel support 442 to rotate relative to the stock 404.

As the break trigger 492 is squeezed, the pivot pin 494 moves proximally. The stop 497 moves proximally as the break trigger 492 is squeezed. As the break trigger 492 is squeezed and the stop 497 moves proximally, the pivot pin 482 can slide within the straight portion 483a of the groove 483 in the base 460 of the stock 404 under applied force by the user. As the break trigger 492 is squeezed and the stop 497 moves proximally, the secondary pivot pin 485 can slide within the curved portion 483b of the groove 483 in the base 460 of the stock 404 under applied force by the user. As the break trigger 492 is squeezed and the stop 497 moves proximally, the guide 487 between the base 454 of the barrel support 442 and the base 460 of the stock 404 can become disengaged. The user can rotate the barrel support 442 clockwise as the pivot pins 482, 485 move within the groove 483. The user can rotate the barrel support 442 about the pivot pins 482, 485. The user can rotate the barrel support 442 and the stock 404 relative to each other.

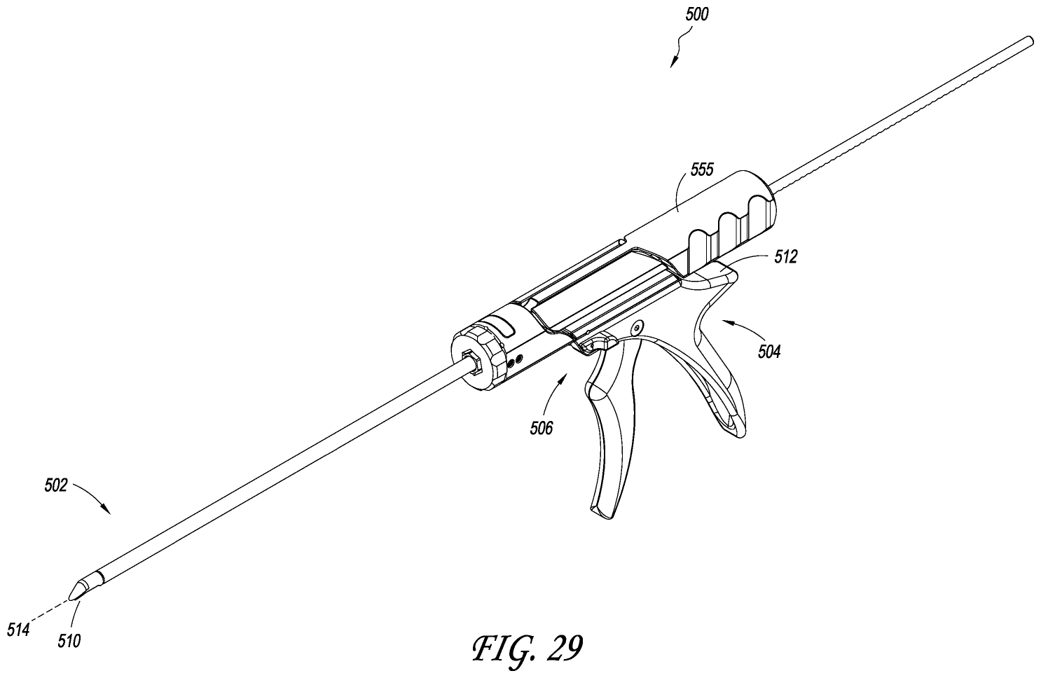

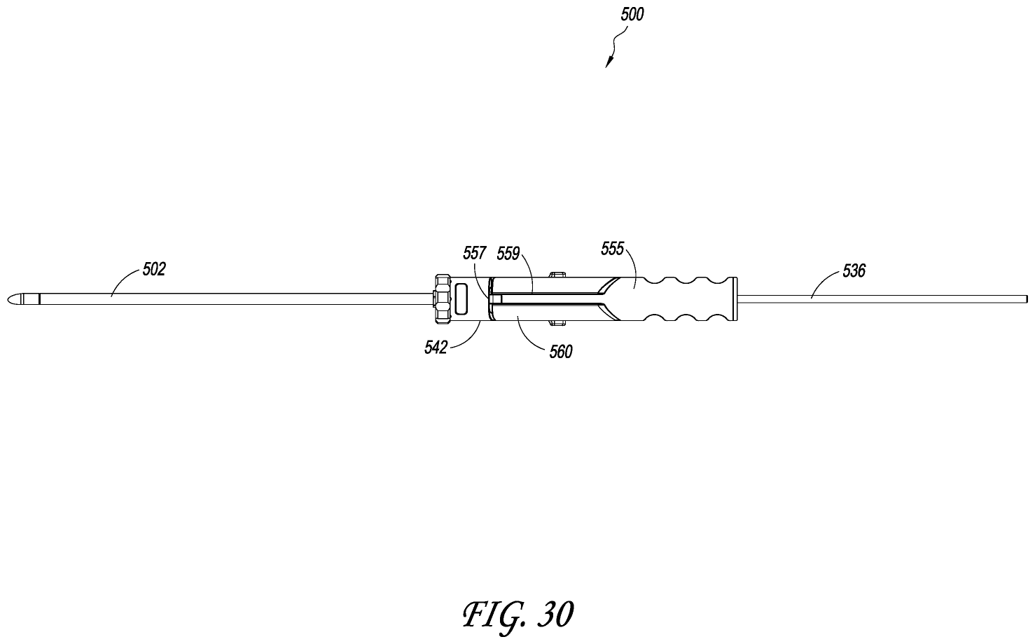

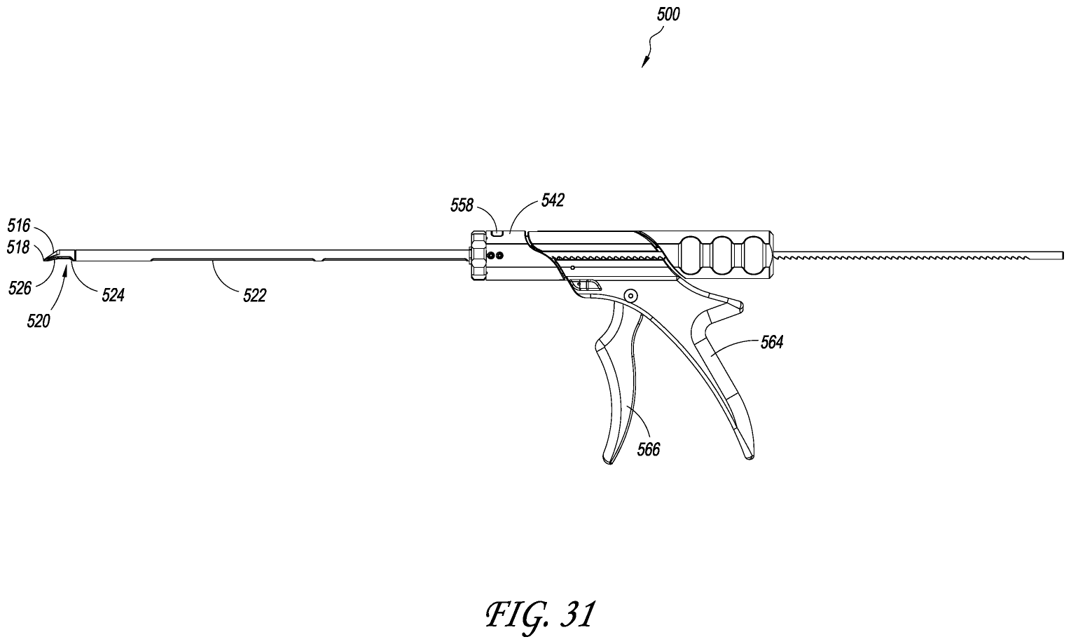

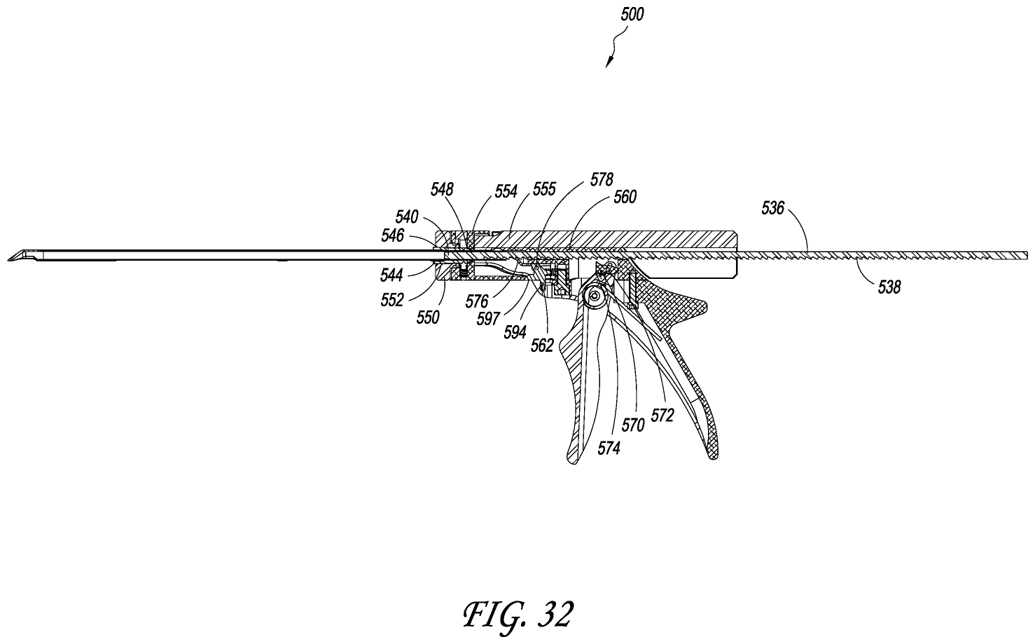

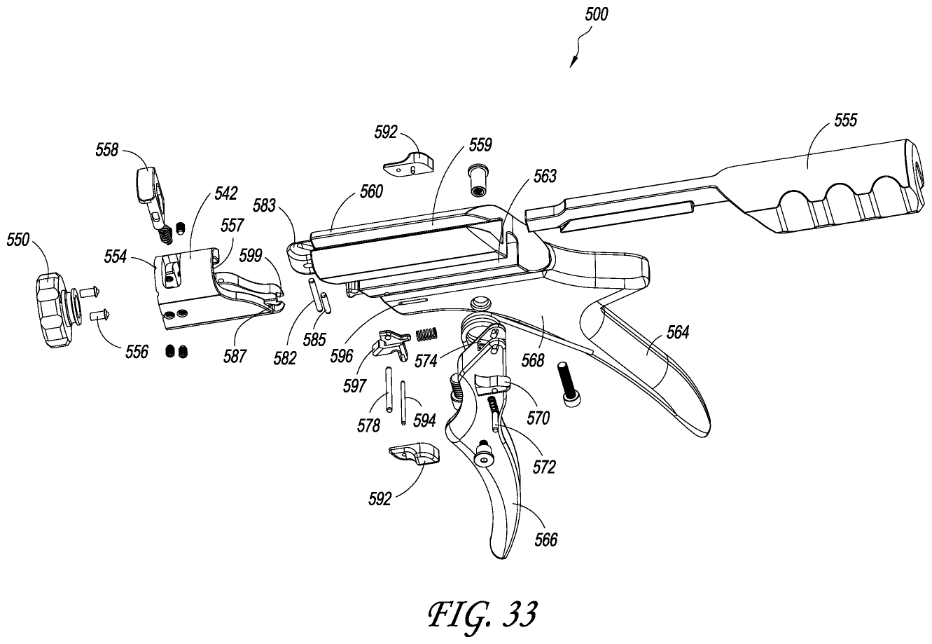

FIG. 29 shows a perspective view of an embodiment of a delivery device 500. FIG. 30 shows a top view of the delivery device 500. FIG. 31 shows a side view of the delivery device 500. FIG. 32 shows a cross-sectional view of the delivery device 500. FIG. 33 is an exploded perspective view of the delivery device 500 with certain components removed. The delivery device 500 can include any of the features of the delivery device 100, 200, 300, 400 described herein.

As shown in FIGS. 29-33, the delivery device 500 can include a barrel 502. The delivery device 500 can include a stock 504. The delivery device 500 can include a break action 506. The delivery device 500 can include a slap hammer 555. The slap hammer 555 allows the delivery device 500 to be impacted. The slap hammer 555 transmits a force through to the barrel 502.

The delivery device 500 can include a distal end 510 and a proximal end 512. The barrel 502 is located near the distal end 510 and the slap hammer 555 is located near the proximal end 512. The delivery device 500 can have a longitudinal axis 514. The longitudinal axis 514 can extend along the barrel 502. The longitudinal axis 514 can extend along the slap hammer 555. The distal end 510 can include an insertion surface 516. The insertion surface 516 can include an atraumatic tip 518.

The barrel 502 can include a lumen 520. The lumen 520 of the barrel 502 is sized to receive a cartridge 530. The cartridge 530 can form a tubular structure. The cartridge 530 can include a distal open end 532 (not shown) and a proximal open end 534. The cartridge 530 is sized to receive a ratchet rod 536. The ratchet rod 536 can include a series of gears 538. The ratchet rod 536 can include a plunger 540. The barrel 502 can include one or more openings 522 to view the cartridge. The distal end 510 can include a delivery opening 524 to expel material from the cartridge 530. The distal end 510 can include a lip 526 to direct the material from the delivery device 500.

The delivery device 500 can include a barrel support 542 to support the barrel 502. The delivery device 500 can include a hub 544 with a keyed section 546 and a groove 548. The hub 544 and the barrel 502 are coupled such that rotation of the hub 544 causes rotation of the barrel 502. The barrel support 542 can include a rotation knob 550. The rotation knob 550 includes a keyed opening 552 to accept the keyed section 546 of the hub 544. The barrel support 542 can include a base 554. The delivery device 500 can include one or more ball plungers 556. The ball plungers 556 can interact with the base 554 to lock the rotational position of the rotation knob 550. The barrel support 542 can include a button 558. Once depressed, the button 558 can allow the hub 544 to disengage from the rotation knob 550 to remove barrel 502. The rotation knob 550 can be rotated to any position, similar to the rotation knobs 150, 250, 350, 450. The base 554 of the barrel support 542 can include a cutout 557. The slap hammer 555 can be shaped to mate with the cutout 557 of the barrel support 542. The slap hammer 555 and the base 554 of the barrel support 542 can form a dovetail joint. The slap hammer 555 and the base 554 of the barrel support 542 can form a friction or interference fit. The slap hammer 555 and the base 554 of the barrel support 542 can form any sliding or removable connection.

The delivery device 500 can include the stock 504. The stock 504 can include a base 560. The base 560 of the stock 504 can include a side slot 563. The base 560 of the stock 504 can include a cutout 559. The slap hammer 555 can be shaped to mate with the cutout 559 of the base 560 of the stock 504. The slap hammer 555 and the base 560 of the stock 504 can form a dovetail joint. The slap hammer 555 and the base 560 of the stock 504 can form a friction or interference fit. The slap hammer 555 and the base 560 of the stock 504 can form any sliding or removable connection. The slap hammer 555 can be utilized to insert the barrel 502 into the surgical site. After insertion of the barrel 502, the slap hammer 555 can be removed from the cutout 557 of the barrel support 542. In some methods, after insertion of the barrel 502, the slap hammer 555 can remain in place in the cutout 557 of the barrel support 542. After insertion of the barrel 502, the slap hammer 555 can be removed from the cutout 559 of the base 560 of the stock 504. In some methods, the slap hammer 555 is removed before activation of the break action 506. In some methods, the slap hammer 555 is rejoined after activation of the break action 506. In some methods, the slap hammer 555 remains in place during activation of the break action 506. The slap hammer 555 can include a slot 561. The slot 561 can be partially enclosed. The slot 561 can be open on a downward side of the slap hammer 555. In some methods, the ratchet rod 536 can extend from the slot 561.

The delivery devices 400, 500 are similar. The delivery device 500 includes a cutout on the superior surface of the delivery device. The cutouts 557, 559 can allow a slap hammer 555 to mate. The slap hammer 555 impacts the internal surface of the delivery device 500 next to the barrel 502 to improve ease of entering the surgical site. The slap hammer 555 may be useful for entering the disc space between vertebral bodies. The slap hammer 555 may be useful for other difficult to access surgical sites. The slap hammer 555 can allow the entire delivery device 500 to be impacted into the disc space and graft extrusion can begin immediately.

The side slot 563 is open to one side of the base 560. In the illustrated embodiment, the side slot 563 is open to the left of the user when the user is holding the delivery device 500 for use. The stock 504 can include a handle 564. The delivery device 500 can include a trigger 566. The delivery device 500 can include a spring 568 to bias the trigger 566.

The delivery device 500 can include a pawl 570. The delivery device 500 can include a pivot pin 572. The pivot pin 572 can extend through the trigger 566 and the pawl 570. The pawl 570 can be disposed within a groove 574 of the trigger 566. The delivery device 500 can include a rocker 576. The delivery device 500 can include a pivot pin 578. The pivot pin 578 can extend through the base 560 of the stock 504 and the rocker 576.

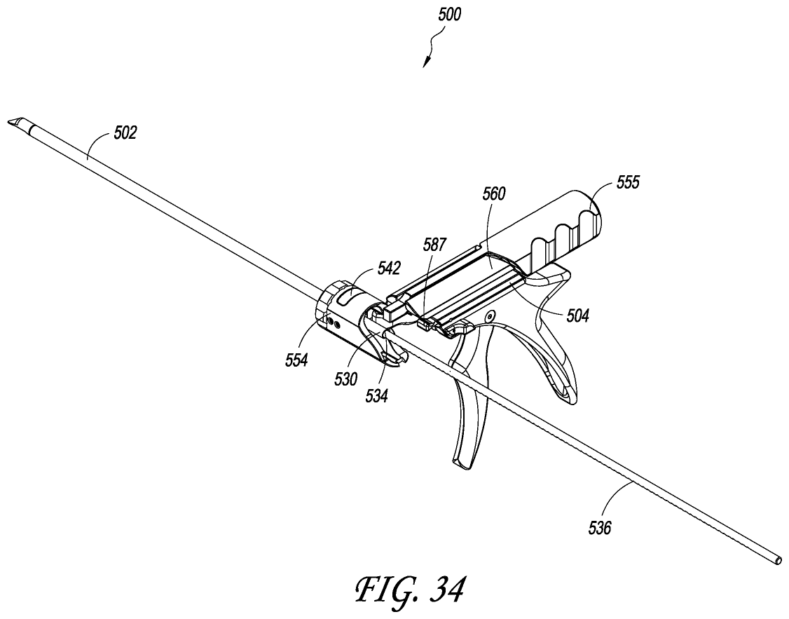

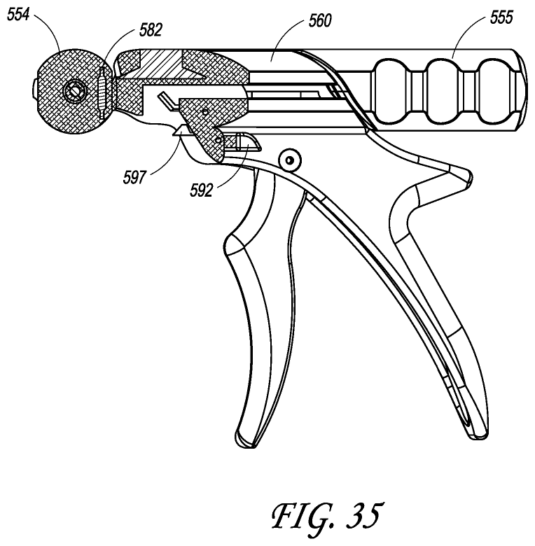

The delivery device 500 can include the break action 506. The break action 506 can include a pivot pin 582. The pivot pin 582 can be disposed between the base 554 of the barrel support 542 and the base 560 of the stock 504. The pivot pin 582 can be disposed perpendicular to the longitudinal axis 514. The pivot pin 582 can be disposed vertically. The pivot pin 582 can be disposed to allow the base 554 of the barrel support 542 to pivot left or right relative to the base 560 of the stock 504. FIG. 34 is a perspective view of the delivery device 500 with the break action 506 activated. FIG. 35 is a cross-sectional view of the delivery device 500 with the break action 506 activated.

The base 560 of the stock 504 can include a groove 583. The groove 583 can include a straight portion 583a and a curved portion 583b. The delivery device 500 can include a secondary pivot pin 585. The pivot pin 582 can extend through the base 554 of the barrel support 542 and within the straight portion 583a. The secondary pivot pin 585 can extend through the base 554 of the barrel support 542 and within the curved portion 583b.

The delivery device 500 can include a guide 587. The guide 587 can be disposed between the base 554 of the barrel support 542 and the base 560 of the stock 504. The guide 587 can include a protrusion on the base 560 of the stock 504 and a corresponding recess on the base 554 of the barrel support 542.

The break action 506 can include a break trigger 592. The delivery device 500 can include a pivot pin 594. The pivot pin 594 is disposed within a groove 596 of the break trigger 592. The pivot pin 594 is disposed within a groove 595 in the handle 564. The break action 506 can include a stop 597. The stop 597 can be integrally formed in the break trigger 592. The stop 597 abuts the base 554 of the barrel support 542. When the stop 297 abuts the base 554 of the barrel support 542, the base 554 of the barrel support 542 is prevented or limited from pivoting left or right relative to the base 560 of the stock 504.

As the break trigger 592 is squeezed, the pivot pin 594 moves proximally. The stop 597 moves proximally as the break trigger 592 is squeezed. As the break trigger 592 is squeezed and the stop 597 moves proximally, the pivot pin 582 can slide within the straight portion 583a of the groove 583 in the base 560 of the stock 504 under applied force by the user. As the break trigger 592 is squeezed and the stop 597 moves proximally, the secondary pivot pin 585 can slide within the curved portion 583b of the groove 583 in the base 560 of the stock 504 under applied force by the user. As the break trigger 592 is squeezed and the stop 597 moves proximally, the guide 587 between the base 554 of the barrel support 542 and the base 560 of the stock 504 can become disengaged. The user can rotate the barrel support 542 clockwise as the pivot pins 582, 585 move within the groove 582. The user can rotate the barrel support 542 about the pivot pins 582, 585.

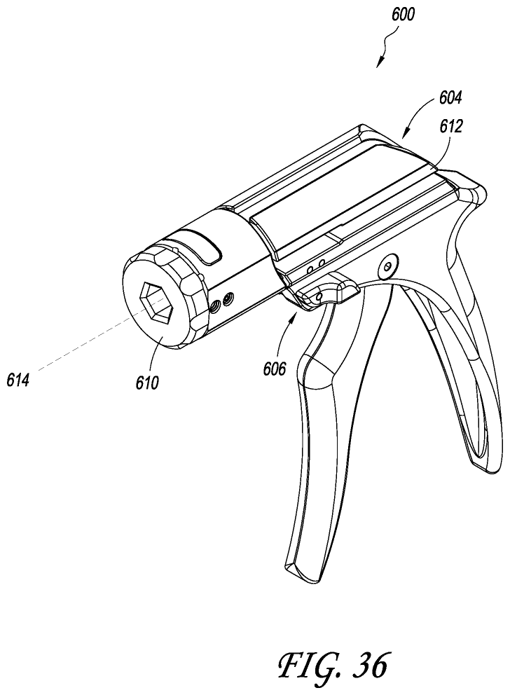

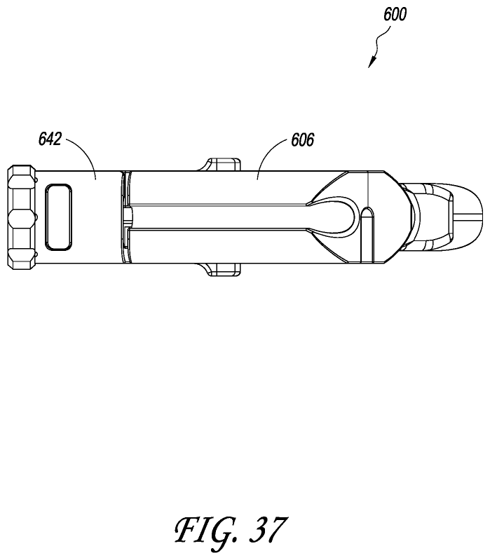

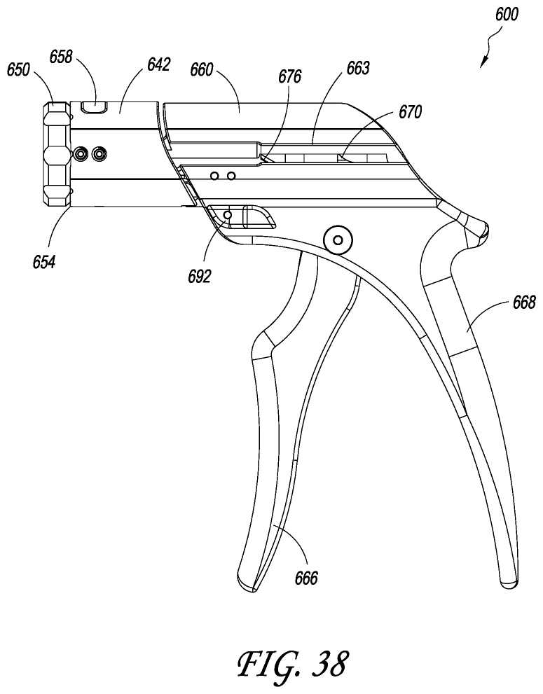

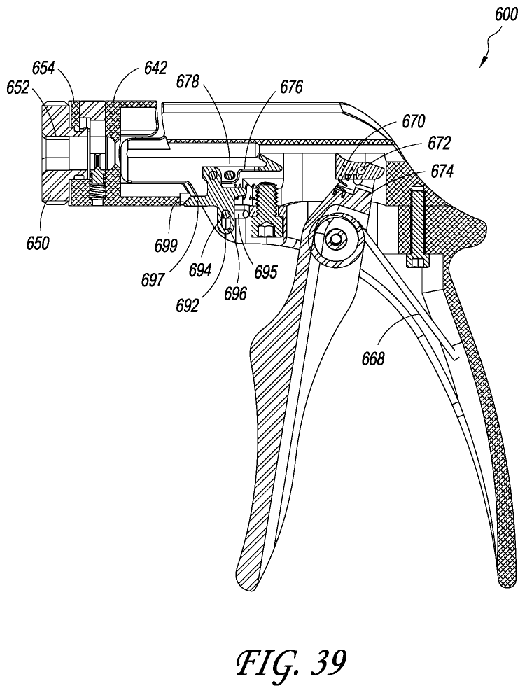

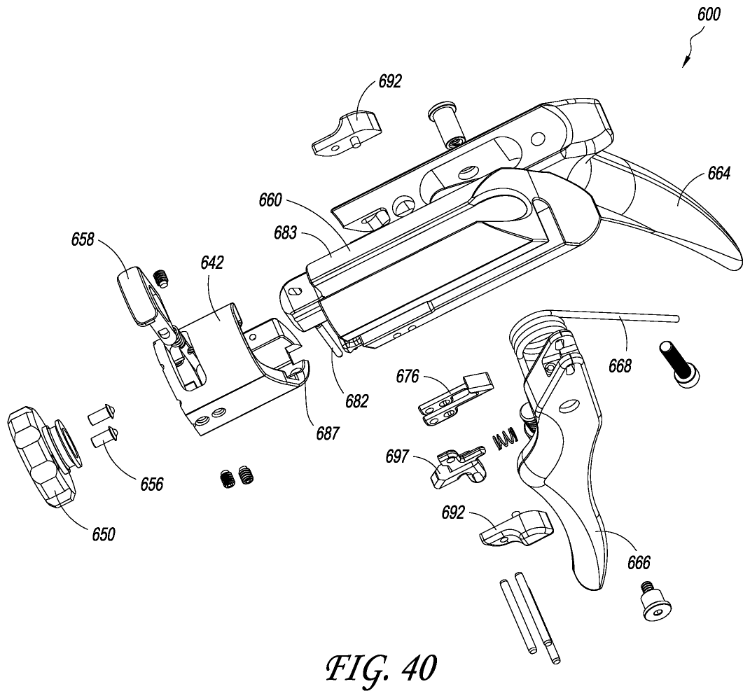

FIG. 36 shows a perspective view of an embodiment of a delivery device 600. FIG. 37 shows a top view of the delivery device 600. FIG. 38 shows a side view of the delivery device 600. FIG. 39 shows a cross-sectional view of the delivery device 600. FIG. 40 is an exploded perspective view of the delivery device 600. The delivery device 600 can include any of the features of the delivery device 100, 200, 300, 400, 500 described herein. The delivery device 600 can have a shorter geometry for the overall delivery device shape.

As shown in FIGS. 36-40, the delivery device 600 can include a barrel 602 (not shown). The barrel 602 can have any features of the barrels 102, 202, 302, 402, 502 described herein. The barrel 602 is sized to receive a cartridge 630 (not shown) and a ratchet rod 636 (not shown). The barrel 602 can be coupled to a hub 644 (not shown). The delivery device 600 can include a stock 604. The delivery device 600 can include a break action 606.

The delivery device 600 can include a distal end 610 and a proximal end 612. The delivery device 600 can have a longitudinal axis 614 extending between the distal end 610 and the proximal end 612.

The delivery device 600 can include a barrel support 642. The barrel support 642 can include a rotation knob 650. The rotation knob 650 includes a keyed opening 652. The keyed opening 652 can be sized to accept the keyed section 646 of the hub 644 (not shown). Rotation of the rotation knob 650 can cause a corresponding rotation of the hub 644, and thus a corresponding rotation of the barrel 602. The barrel support 642 can include a base 654. The rotation knob 650 includes one or more ball plungers 656. The ball plungers 656 interact with the base 654 to lock the rotational position of the barrel 602. The barrel support 642 can include a button 658. The button 658 can allow the hub 644 to disengage from the rotation knob 650 to remove barrel 602. The cartridge 630 enters the barrel support 642 of the delivery device 600. In some embodiments, the cartridge 630 enters the stock 604 of the delivery device 600.

The delivery device 600 can include the stock 604. The stock 604 can include features for a compact or low profile design. The stock 604 can include a base 660. The base 660 can be shorter in length than the base 160, 260, 360, 460, 560. The length can be a measurement along the longitudinal axis 614. The base 660 of the stock 604 can include a side slot 663. The side slot 663 is open to one side of the base 660. In the illustrated embodiment, the side slot 663 is open to the left when the user is holding the delivery device 600 for use. The stock 604 can include a handle 664. The handle 664 can be shorter in length than the handle 164, 264, 364, 464, 564. The delivery device 600 can include a trigger 666. The delivery device 600 can include a spring 668 to bias the trigger 666. The delivery device 600 can include a barrel 602. The barrel 602 can be shorter in length than the barrel 102, 202, 302, 402, 502.

The delivery device 600 can include a pawl 670. The pawl 670 can have a smaller, compact shape than the pawl 170, 270, 370, 470, 570. Any size or shape pawl is contemplated that functions to engage the ratchet rod 636. The delivery device 600 can include a pivot pin 672. The pivot pin 672 can extend through the trigger 666 and the pawl 670. The pawl 670 can be disposed within a groove 674 of the trigger 666. The delivery device 600 can include a rocker 676. The delivery device 600 can include a pivot pin 678. The pivot pin 678 can extend through the base 660 of the stock 604 and the rocker 676. The rocker 676 can have a smaller, compact shape than the rocker 176, 276, 376, 476, 576. The rocker 676 can be disposed in the opposite direction than the rocker 176, 276, 376, 476, 576. The rocker 676 can have an acute included angle. The rocker 176, 276, 376, 476, 576 can have an obtuse included angle. Any size or shape rocker is contemplated that functions to engage the ratchet rod 636. The rocker 676 can be a leaf spring. The rocker 676 extends behind the mounting point. The rocker 176, 276, 376, 476, 576 can extend in front of the mounting point. The rocker 676 operates in the same manner to prevent the ratchet rod 636 from travelling backwards. The rocker 676 is shorter than the rocker 176, 276, 376, 476, 576. The rocker 676 extends behind the pivot point. The rocker 676 extends at an acute angle. The rocker 176, 276, 376, 476, 576 extends forward with an obtuse angle.

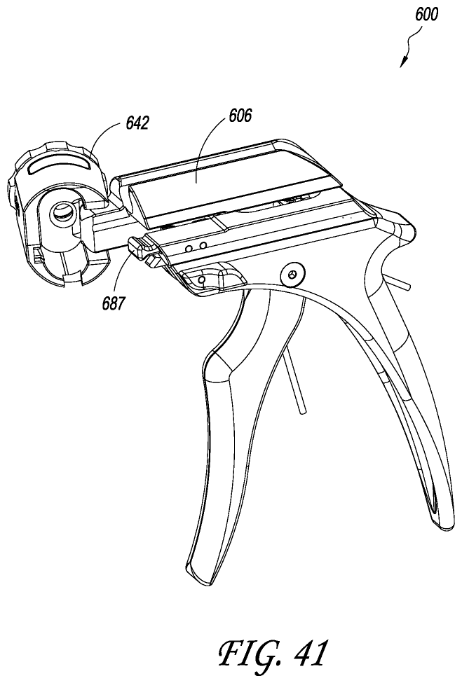

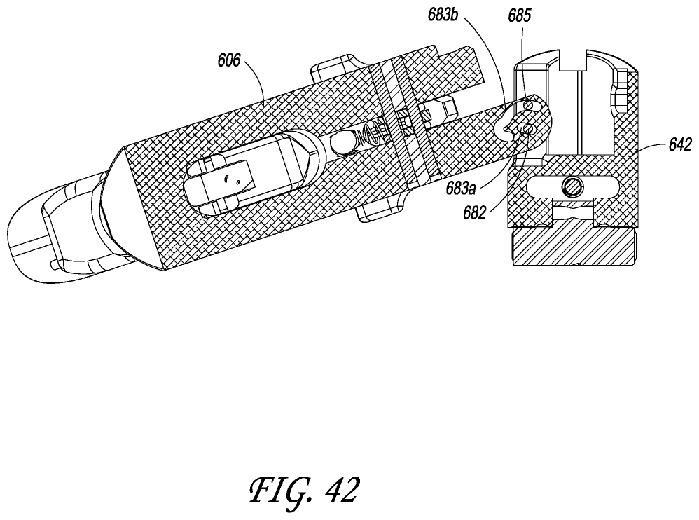

The delivery device 600 can include the break action 606. The break action 606 allows for loading of the cartridge 630 within the barrel 602. The break action 606 can include a pivot pin 682. The pivot pin 682 can be disposed between the base 654 of the barrel support 642 and the base 660 of the stock 604. The pivot pin 682 can be disposed perpendicular to the longitudinal axis 614. The pivot pin 682 can be disposed to allow the base 654 of the barrel support 642 to pivot left or right relative to the base 660 of the stock 604. FIG. 41 is a perspective view of the delivery device 600 with the break action 606 activated. FIG. 42 is a cross-sectional view of the delivery device 600 with the break action 606 activated.

The base 660 of the stock 604 can include a groove 683. The groove 683 can include a straight portion 683a and a curved portion 683b. The delivery device 600 can include a secondary pivot pin 685. The pivot pin 682 can extend through the base 654 of the barrel support 642 and within the straight portion 683a. The secondary pivot pin 685 can extend through the base 654 of the barrel support 642 and within the curved portion 683b.

The pivot of the delivery device 600 is a cam centered around a slanted slot. The pivot of the delivery device 600 can include the straight portion 683a. The straight portion 683a can provide the advantage of pulling the barrel support 642 and the stock 604 together under axial load. The pivot of the delivery device 600 can include the curved portion 683b. When opening and closing the delivery device 600, the curved portion 683b guides the secondary pivot pin 685 to ensure smooth opening and closing. The secondary pivot pin 685 functions as a cam.

When opening and closing the delivery device 600, the curved portion 683b avoids unintentional engagement of a guide 687 opposite the off center pivot. The pivot of the delivery device 600 can include the guide 687. This axial loading engages the guide 687 on the opposite side of the delivery device 600 as the pivot. The guide 687 can prevent the delivery device 600 from opening up due to the off center pivot. The guide 687 can be disposed between the base 654 of the barrel support 642 and the base 660 of the stock 604. The guide 687 can include a protrusion on the base 660 of the stock 604 and a corresponding recess on the base 654 of the barrel support 642. The guide 487 can prevent the pivot from opening inadvertently.

The break action 606 can include a break trigger 692. In some embodiments, the rocker 676 is coupled to the break trigger 692. The rocker 676 can be coupled to the break trigger 692 for a compact design of the delivery device 600. In other embodiments, the rocker 676 and the break trigger 692 are not coupled.

The delivery device 600 can include a pivot pin 694. The pivot pin 694 is disposed within a groove 696 of the break trigger 692. The pivot pin 694 is disposed within a groove 695 in the handle 664. The break action 606 can include a stop 697. The stop 697 can be integrally formed in the break trigger 692. The stop 697 abuts the base 654 of the barrel support 642. When the stop 697 abuts the base 654 of the barrel support 642, the base 654 of the barrel support 642 is prevented or limited from pivoting left or right relative to the base 660 of the stock 604.

As the break trigger 692 is squeezed, the pivot pin 694 moves proximally and the stop 697 moves proximally. As the break trigger 692 is squeezed and the stop 697 moves proximally, the pivot pin 682 can slide within the straight portion 683a of the groove 683 in the base 660 of the stock 604 and the secondary pivot pin 685 can slide within the curved portion 683b of the groove 683 in the base 660 of the stock 604. As the break trigger 692 is squeezed and the stop 697 moves proximally, the guide 687 between the base 654 of the barrel support 642 and the base 660 of the stock 604 can become disengaged. The user can rotate the barrel support 642 relative to the stock 604.

The delivery devices can have several advantages. The delivery devices can place material at a surgical site. In some embodiments, the delivery device opens in line with the barrel. In some embodiments, the delivery device has a break action. In some embodiments, the delivery device includes a break action along a centerline. In some embodiments, the delivery device includes a break action that opens a latch. In some embodiments, the delivery device includes a break action that allows controlled pivoting in one direction. In some embodiments, the delivery device includes a break action that limits the range of pivoting. In some embodiments, the delivery device includes a break action that limits the direction of pivoting.

In some embodiments, the delivery device advances material with a pawl and ratchet. In some embodiments, the delivery device prevents back-out of the ratchet. In some embodiments, the delivery device includes one or more ejectors. In some embodiments, the delivery device includes a side slot. In some embodiments, the delivery device includes a compact design. In some embodiments, the delivery device includes a combination of a rocker and break action. In some embodiments, the delivery device includes a combination of trigger and pawl. In some embodiments, the delivery device includes a shortened length.

In some embodiments, the cartridge can be loaded before the tip is inserted into the surgical site. In some embodiments, the cartridge can be unloaded after the tip is inserted into the surgical site. In some embodiments, another cartridge can be loaded after the tip is inserted into the surgical site. In some embodiments, the cartridge can be loaded with a line of sight to the barrel. In some embodiments, the cartridge can be unloaded with a line of sight to the barrel. In some embodiments, the break action allows for easy loading of the cartridge. In some embodiments, the break action allows for easy unloading of the cartridge. In some embodiments, the break action allows for easy reloading of the cartridge. In some embodiments, the break action allows for unloading a spent cartridge and reloading a new cartridge without removing the tip from the surgical site.

In some embodiments, the delivery device can be actuated with a single hand of the user. In some embodiments, the user can grip the handle and the trigger with the single hand. In some embodiments, the user can move the index finger of the hand to activate the break trigger. In some embodiments, the user can pivot the stock relative to the barrel support. In some embodiments, the user can pivot the barrel support relative to the stock. In some embodiments, the user can load the cartridge. In some embodiments, the delivery device allows for ambidextrous use. In some embodiments, the delivery device can be a mirror image of any delivery device described herein, to accommodate an alternate side to side action, top to bottom action, or other action of the delivery device. In some embodiments, the delivery device can include any of the features of the various embodiments described herein.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. For all the embodiments described above, the steps of the methods need not be performed sequentially. Thus, it is intended that the scope of the present invention herein disclosed should not be limited by the particular disclosed embodiments described above, but should be determined only by a fair reading of the claims that follow.

What is claimed is:

1. A delivery device comprising:
a barrel support configured to couple with a barrel;
a stock comprising a handle; and
wherein the stock is configured to pivot relative to the barrel support when the barrel is positioned at a surgical site, wherein the pivoting is configured to facilitate positioning a cartridge within a lumen of the barrel.

2. The delivery device of claim 1, wherein the barrel support comprises a rotation knob configured to allow the barrel to rotate relative to the barrel support.

3. The delivery device of claim 1, further comprising a horizontal pivot pin configured to allow upward and downward motion of the stock relative to the barrel support.

4. The delivery device of claim 1, further comprising a vertical pivot pin configured to allow side to side motion of the stock relative to the barrel support.

5. The delivery device of claim 1, further comprising a trigger configured to allow the stock to pivot.

6. The delivery device of claim 1, further comprising a slide mechanism configured to allow the stock to pivot.

7. The delivery device of claim 1, further comprising at least one pin configured to slide within a groove of a guide when the stock pivots.

8. The delivery device of claim 1, further comprising an ejector flange configured to slide relative to a flange of a cartridge.

9. The delivery device of claim 1, further comprising a pin configured to slide out of an interference fit with a lumen of the barrel support to allow the stock to pivot.

10. The delivery device of claim 1, further comprising a stop configured to slide out of engagement with the barrel support to allow the stock to pivot.

11. The delivery device of claim 1, further comprising a pawl and a ratchet rod to advance material through the cartridge.

12. The delivery device of claim 1, further comprising a slap hammer configured to couple with the stock.

13. The delivery device of claim 1, further comprising a trigger to advance material through the cartridge.

14. A method of delivering material to a surgical site, the method comprising:
inserting a tip of a barrel of a delivery device into a surgical site, the barrel coupled to a barrel support, the barrel support coupled to a stock comprising a handle;
pivoting the stock relative to the barrel support when the barrel is positioned at the surgical site; and
inserting a cartridge within a lumen of the barrel when the stock is pivoted.

15. The method of claim 14, further comprising removing the cartridge from the lumen of the barrel.

16. The method of claim 15, further comprising inserting a second cartridge into the lumen of the barrel.

17. The method of claim 14, wherein pivoting the stock relative to the barrel support further comprises pulling a trigger with the index finger of a hand of a user.

18. The method of claim 17, further comprising holding the handle with the hand of the user while actuating the trigger.

19. The method of claim 14, wherein the stock is pivoted upward and downward relative to the barrel support.

20. The method of claim 14, wherein the stock is pivoted side to side relative to the barrel support.
